(19) United States
(12) Patent Application Publication
(10) Pub. No.: US 2022/0226310 A1
(43) Pub. Date: Jul. 21, 2022

(54) Bio-Based Medicines and Methods of Increasing Patient Compliance

(71) Applicant: Anellotech, Inc., Pearl River, NY (US)

(72) Inventor: Sudolsky David, Pearl River, NY (US)

(21) Appl. No.: 17/607,352

(22) PCT Filed: May 28, 2019
(86) PCT No.: PCT/US19/34227

§ 371 Date: Oct. 28, 2021

Related U.S. Application Data
(60) Provisional application No. 62/677,161, filed on May 28, 2018.

(51) Int. Cl. A61K 31/495; A61K 31/137; A61K 31/4439; A61K 45/06

(52) U.S. Cl. CPC A61K 31/495; A61K 45/06; A61K 31/4439; A61K 31/137

(57) ABSTRACT

Medicines that are used to treat diseases are conventionally made from starting materials that are derived from fossil fuels and therefore contain essentially no ¹⁴C. In the present invention, medicines can be partially or fully derived from biological sources and therefore contain about one part per trillion (ppt) ¹⁴C. These compounds have been discovered as superior for the treatment of disease because they have surprisingly been found to substantially increase patient compliance. Compounds and methods of using partially or fully bio-based pharmaceutically active compounds to track metabolites are also disclosed.

### RELATED APPLICATIONS

This application claims the priority benefit of U.S. Provisional Patent Application Ser. No. 62/677,161, filed 28 May 2018.

### BACKGROUND

To be optimally effective, most medications require scheduled use over a period of time. Whether people follow this regimen is termed “compliance” or, equivalently, “adherence,” and the failure of people to comply or adhere to the regimen is known to be a significant problem. Speaking generally of patients, and with reference to three earlier publications, McElnay et al. in “Self-reported medication non-compliance in the elderly,” Eur. J. Clin Pharmacol (1997) 53: 171-178 state “It is now widely accepted that, in general terms, one third of patients comply ‘partially,’ taking between 40% and 80% of doses; one third comply ‘satisfactorily’, occasionally taking more, occasionally taking less of the prescribed amount; one sixth take less than 40% of the prescribed doses with widely varying intervals; while one sixth are good compliers.”

In view of this problem, there is a strong incentive to provide new solutions to improve patient compliance.

### SUMMARY

Pharmaceutically active compounds are disclosed that are based on known structures wherein the structures are fully or partially derived from biomass such that the ¹⁴C content in the structure or selected moiety is similar to the ¹⁴C content in living organisms, on the order of 1 part per trillion. Also disclosed are methods of treating a patient comprising a step of administering (or prescribing) one or more of the above-described bio-based pharmaceutical compounds or compositions to a patient. Preferably, the patient is aware of the bio-based nature of the drug composition. The disclosed compounds have a higher percentage of bio-based carbon (that is, a higher ¹⁴C/¹²C isotopic ratio) than is present in fossil-based compounds.

In one aspect, the invention provides a pharmaceutically active compound that is at least partially derived from biomass. The pharmaceutically active compound that is at least partially derived from biomass can be any of the compounds in Table 1; especially preferred examples include: Chlorhexidine (chlorhexamed forte), Ambroxol (mucosolvan), Cetirizine (Hexal), Bisacodyl (Ducolax), Xylomethazoline (Olynth), Diclofenac (used to treat pain and inflamatory diseases)(forte voltaren), Clotrimazole (canesten), Omeprazole (omep Hexal), Flurbiprofen (to treat pain and arthritis) (Dobendan), Naproxen (Dolormin), Doxilamine (Hoggar), Ioperamide, Ibuprofen, or lansoprazole. In some preferred embodiments, the pharmaceutically active compound has one or more of the following characteristics: comprising at least one aromatic group that is derived from biomass; where all the aromatic groups in the active compound are derived from biomass; where the entire compound is derived from biomass; the pharmaceutically active compound having a ¹⁴C:¹²C isotopic ratio that is similar to the ¹⁴C:¹²C isotopic ratio of a living organism (approximately 1 part per trillion); the compound can be pure or in a mixture such as with one or more pharmaceutically acceptable excipient and/or in a mixture comprising at least two pharmaceutically active compounds; the compound contains at least 10%, at least 40%, or at least 50%, or at least 70%, or 100%, or between 10 and 90%, or between 40 and 90%, or between 50 and 90 mass % bio-based carbon (percentages are always in mass unless indicated otherwise); the specific compounds listed above may be characterized by carbon ratios characteristic of the synthesis; the bio-based carbon in any of the foregoing percentages may be derived from plants (which may be termed raw plant materials or “environmentally-friendly renewable raw plant materials”); composition is in medicine delivery form such as tablet, syrup, IV bag, or capsule; the compound is in a composition that comprises at least 1 mg, or at least 5 mg, or at least 10 mg, or at least 40 mg of the active compound in a medicine delivery form; the composition comprises at least 1%, 2% or at least 10%, or at least 50%, or at least 80% by mass of the active compound; and/or wherein the compound or composition is characterizable by an increase in compliance of at least 10%, or at least 30%, or between 20 and 67%, or between 20 and 58%, or between 10 and 37%, or between 10 and 28%, or between 20 and 28%.

In an alternative aspect, the invention can be described as a substance X for use in improving patient compliance with a pharmaceutical dosing regime, wherein substance X is one of Chlorhexidine (chlorhexamed forte), Ambroxol (mucosolvan), Cetirizine (Hexal), Bisacodyl (Ducolax), Xylomethazoline (Olynth), Diclofenac (used to treat pain and inflamatory diseases)(forte voltaren), Clotrimazole (canesten), Omeprazole (omep Hexal), Flurbiprofen (to treat pain and arthritis) (Dobendan), Naproxen (Dolormin), Doxilamine (Hoggar), Ioperamide, and Ibuprofen; and wherein substance X comprises at least 10 mass % of bio-based carbon. Preferably, at least 40%, or at least 50%, or at least 70%, or 100% bio-based carbon. Likewise, this substance can additionally, have one or any combination of the characteristics described above or in the detailed description section below. In broader aspects, the substance can be any of the compounds in Table 1.

In another aspect, the invention provides a pharmaceutically active compound in which between 10 and 90 mass % of the carbon atoms are bio-based. The compound may be selected from Table 1. Compound is Chlorhexidine (chlohexamed forte), Ambroxol (mucosolvan), Cetirizine (Hexal), Bisacodyl (Ducolax), Xylomethazoline (Olynth), Diclofenac (forte voltaren), Clotrimazole (canesten), Omeprazole (omep Hexal), Flurbiprofen (Dobendan), Naproxen (Dolormin), Doxilamine (Hoggar), Ioperamide, or Ibuprofen. Preferably, at least 40%, or at least 50%, or at least 70%, of the carbons in the active compound is bio-based carbon. The compound can be used to study metabolism of drug as compared to a conventional non-bio-based drug by assessing metabolites, transport, and/or distribution of ¹⁴C-containing compounds or moieties.

The invention also includes methods of treating a disease state comprising administering to patient in need thereof, a composition comprising a pharmaceutically active compound that is at least partially derived from biomass and, optionally, having one or any combination of the above characteristics. In preferred embodiments, the patient knows that the at least one pharmaceutically active compound is at least partially derived from biomass. Preferably, the compound is administered in a dosage regimen comprising multiple doses administered (in some preferred embodiments, self-administered) over a period of at least 3 days, or at least 5 days, or at least 10 days, or at least 30 days, in some embodiments between 3 and 30 days. The methods of treating the disease state preferably improve patient compliance as compared with conventionally-derived (i.e., derived from fossil fuels) pharmaceuticals.

In a related aspect, the invention provides a method of improving patient compliance with a pharmaceutical dosing regime, comprising administering a pharmaceutically active compound that is at least partially derived from biomass in the dosing regime.

In some aspects, the invention provides a pharmaceutically active compound that is at least partially derived from biomass for treating a disease state: Chlorhexidine for treating infections or for tracking metabolism, Ambroxol for treatment of respiratory diseases, Cetirizine for the treatment of allergy symptoms, Bisacodyl to treat constipation, Xylomethazoline to treat nasal congestion, Diclofenac to treat pain and inflamatory diseases, Clotrimazole to treat fungal infections, Omeprazole to treat stomach ulcers and acid reflux, Flurbiprofen to treat pain and arthritis, Naproxen to treat fever and pain, Doxilamine to treat allergy symptoms, Ioperamide to treat diarrhea, and Ibuprofen to treat fever and pain, lansoprazole for treating stomach ulcers, a damaged esophagus, gastroesophageal reflux disease (GERD), or high levels of stomach acid, or mephentermine for treatment of low blood pressure.

In another aspect, the pharmaceutically active compound is used to study metabolism of drug as compared to a conventional non-bio-based drug by assessing metabolites, transport, and/or distribution of ¹⁴C-containing compounds or moieties. The invention provides a method of assessing the metabolism of a pharmaceutically active compound in a patient population, including the steps of (i) administering to patients in the patient population a pharmaceutically active compound that is at least partially derived from biomass and (ii) assessing the isotopic ratio of at least one metabolite of the pharmaceutically active compound.

In a further aspect, the invention provides a method of making a biomass-based pharmaceutically active compound comprising reacting a biomass-based aromatic with another organic molecule to yield an at least partially biomass based pharmaceutically active molecule.

The compounds, compositions, and methods disclosed herein provide certain advantages over the art, including increased patient acceptance of the drug product and patient compliance. It is a utility of the disclosed compounds, compositions, and methods, that with patient knowledge that the drug product or its constituent pharmaceutically active compound is sourced from natural feedstocks such as biomass, preferably plants, (including but not limited to, wood, corn stover, sugar cane bagasse, other agricultural resources), patient compliance improves. Due to increased patient confidence in bio-based cures, the disclosed compounds, compositions and methods lead to higher patient compliance (with better effectiveness and reduced recurrence of symptoms in certain cases) and, thus, better patient outcomes.

An additional, and distinct, advantage of the disclosed compounds, compositions, and methods is that any of the disclosed bio-based compounds can be used in radio-labeled studies. For example, such studies are useful in the study of the metabolism of pharmaceutically active compounds and drug products. The ¹⁴C metabolites and moieties can be traced as they move and/or change as they interact with a living organism. Partially bio-based pharmaceutically active compounds may be especially useful in tracing moieties as the compound is interacting in a biological system and metabolized. The drug structures may be fully bio-based or only partially bio-based where only a portion (typically the aryl group(s)) is bio-based so that different metabolites have different ¹⁴C/¹²C ratios.

### Glossary

Aromatics—As used herein, the terms “aromatics” or “aromatic compound” are used to refer to a hydrocarbon compound or compounds comprising one or more aromatic groups such as, for example, single aromatic ring systems (e.g., benzyl, phenyl, etc.) and fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene), naphthalene, methyl-naphthalene (e.g., 1-methyl naphthalene), anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dimethyl-hydrindene. Single-ring and/or higher ring aromatics may also be produced in some embodiments. Aromatics also include single and multiple ring compounds that contain heteroatom substituents, i.e. phenol, cresol, benzofuran, aniline, indole, etc.

Biomass—As used herein, the term “biomass” is given its conventional meaning in the art and is used to refer to any organic source of energy or chemicals that is renewable. Its major components can be: (1) trees (wood) and all other vegetation; (2) agricultural products and wastes (corn, fruit, garbage ensilage, etc.); (3) algae and other marine plants; (4) metabolic wastes (manure, sewage), and (5) cellulosic urban waste. Examples of biomass materials are described, for example, in Huber, G. W. et al, “Synthesis of Transportation Fuels from Biomass: Chemistry, Catalysts, and Engineering,” Chem. Rev. 106, (2006), pp. 4044-4098.

Biomass has been defined as the living and recently dead biological material that can be converted for use as fuel or for industrial production. The criterion as biomass is that the material should be recently participating in the carbon cycle so that the release of carbon in the combustion process results in no net increase averaged over a reasonably short period of time (for this reason, fossil fuels such as peat, lignite and coal are not considered biomass by this definition as they contain carbon that has not participated in the carbon cycle for a long time so that their combustion results in a net increase in atmospheric carbon dioxide). Most commonly, biomass refers to plant matter grown for use as biofuel, but it also includes plant or animal matter used for production of fibers, chemicals or heat. Biomass may also include biodegradable wastes or byproducts that can be burnt as fuel or converted to chemicals, including municipal wastes, green waste (the biodegradable waste comprised of garden or park waste, such as grass or flower cuttings and hedge trimmings), byproducts of farming including animal manures, food processing wastes, sewage sludge, and black liquor from wood pulp or algae. Biomass excludes organic material which has been transformed by geological processes into substances such as coal, oil shale or petroleum. Biomass is widely and typically grown from plants, including miscanthus, spurge, sunflower, switchgrass, hemp, corn (maize), poplar, willow, sugarcane, and oil palm (palm oil) with the roots, stems, leaves, seed husks and fruits all being potentially useful. Biomass can be distinguished from fossil-derived carbon by the presence of ¹⁴C in amounts significantly above that found in fossil fuels.

“Bio-based” means that the carbon in the drug structure or a selected part of the drug structure has been derived from biomass such that the ¹⁴C content in the structure or selected moiety is similar to the ¹⁴C content in living organisms, on the order of 1 part per trillion. The ¹⁴C content can be measured by radiation counting or accelerator mass spectrometry.

Catalytic pyrolysis refers to a process for converting hydrocarbonaceous materials to chemicals, fuels, or chemicals and fuels by rapid heating in the presence of a catalyst. Examples of apparatus and process conditions suitable for CFP are described in U.S. Pat. Nos. 8,277,643, and 9,169,442, by Huber et al., and in US Patent Application 2013/0060070A1 by Huber et al. that are incorporated herein by reference. Conditions for catalytic pyrolysis of biomass may include one or any combination of the following features (which are not intended to limit the broader aspects of the invention): a zeolite catalyst, a ZSM-5 catalyst; a zeolite catalyst comprising one or more of the following metals: titanium, vanadium, chromium, manganese, iron, cobalt, nickel, copper, zinc, gallium, platinum, palladium, silver, phosphorus, sodium, potassium, magnesium, calcium, tungsten, zirconium, cerium, lanthanum, and combinations thereof; a fluidized bed, circulating bed, or riser reactor; an operating temperature in the range of 300° to 1000° C.; and/or a solid catalyst-to-biomass mass ratio of between 0.1 and 40.

“Compliance,” is a widely understood term which is also known as “adherence,” and refers to the extent to which a patient adheres to a dosing regimen. This is equivalent to the extent to which patients administer a drug product consistently for the prescribed amount of medicine for the prescribed time interval over the course of treatment. Here “prescribed” may mean the prescription of a medical professional (typically a doctor or nurse) or labeled instructions on an over-the-counter medication. Patient compliance for many drug regimens is known to be poor, and even in many cases of drug products for the treatment of life-threatening diseases patient compliance is as low as 50%. Compliance can be measured by conventional means, for example, asking patients about their administration, or testing their urine or blood. For purposes of the present invention, compliance can be measured by asking prospective patients about their compliance under a given set of circumstances, or by asking people, preferably users of the medicine, about the compliance of users generally under a given set of circumstances. In some embodiments, changes in compliance can be calculated, for example, by adding categories such as more likely and much more likely to comply with a dosage regimen minus less likely or much less likely (see FIG. 2).

A dosage regimen is the schedule of doses of a medicine, including the time between doses, the duration of treatment and the amount to be taken each time. Dosage regimens also include how a medicine is to be taken, and in what formulation (dosage form). This is the conventional definition and is the definition found in the European's Patient Academy since at least 2016.

As is standard patent terminology, the term “consisting essentially of” excludes the presence of additional steps that would materially affect the method or components that would materially affect the product. In general, any of the inventive methods or products that are defined using the term “comprising” may also be characterized using the more restrictive term “consisting essentially of” or, in the narrowest case, “consisting of.”

### BRIEF DESCRIPTION OF THE DRAWINGS

Table 1 is a listing of small molecule drugs.

FIG. 1 is a graph summarizing survey data that shows the percentage of consumers indicating the percentage of plant-based ingredients in a product that would cause them to buy the bio-based medicine.

FIG. 2 shows the increase in compliance for bio-based cetirizine anticipated by consumers in Germany, Sweden and the United Kingdom.

FIG. 3 shows the increase in compliance for bio-based ibuprofen anticipated by consumers in Germany as compared to conventionally-sourced ibuprofen.

### DETAILED DESCRIPTION

In the present invention, bio-based medicines are synthesized from starting materials that are sourced from renewable sources (as opposed to fossil fuels). There are numerous patents and papers describing methods of making bio-based materials from renewable sources. Preferred starting materials for making pharmaceutical compositions according to the present invention are the aromatic products made by pyrolysis of biomass as described in the Huber patents cited above. To mention another example, Miller et al. in U.S. Pat. No. 9,668,951 (incorporated herein as if reproduced in full below) describe making bio-based 1,3-propanediol in a microbial process. Cukalovic in “Use of microreactor technology and renewable resources to develop green chemical processes,” Ph.D. dissertation, Ghent University, 2012 describes reductive amination of hydroxymethylfuran (HMF) resulting in (5-alkylaminomethyl-2-hydroxmethyl)furan structures that can be converted into 6-substituted 3-pyridinols useful in sensory research or starting materials for further conversions, into various pharmaceuticals or agrochemicals (citing Kohl et al, “The Selection of Pantoprazole as a clinical Candidate,” J. Med. Chem. (1992), vol. 35, Issue 6, pages 1049-1057). Tsolakis et al., in Mapping supply dynamics in renewable feedstock enabled industries: A systems theory perspective on “green” pharmaceuticals, Operations Management Research (2018), Vol. 11, pages 83-104 report that, for the case of paracetamol, an active pharmaceutical ingredient (API) could be manufactured from terpenoid feedstocks, either limonene or β-pinene. The identification of suppliers of limonene-found in significant concentrations in citrus waste—or β-pinene—extracted in substantial volumes from crude sulphate turpentine found in waste from kraft paper and pulp industries. Mahmoud in The selective synthesis of aromatics and furans from biomass-derived compounds, Thesis, 2016, University of Delaware mentions that the Diels-Alder reaction of furans is an important reaction for the conversion of these compounds to aromatic molecules, the synthesis of pharmaceuticals, and a variety of other important molecules. Other publications describing bio-based substances include: Xu et al., Direct production of indoles via thermos-catalytic conversion of bio-derived furans with ammonia over zeolites, Green Chemistry (2015), Vol. 17, pages 1281-1290; Carlson et al., Aromatic Production from Catalytic Fast Pyrolysis of Biomass-derived Feedstock, Topics in Catalysis (2004), vol. 52, pages 241-252.

Testing methods for bio-based carbon are well known. ASTM D6866-18, Entitled Standard Test Methods for Determining the Biobased Content of Solid, Liquid, and Gaseous Samples Using Radiocarbon Analysis, provides accurate biobased/biogenic carbon content results to materials whose carbon source was directly in equilibrium with C02 in the atmosphere at the time of cessation of respiration or metabolism, such as the harvesting of a crop or grass living its natural life in a field. Liquid Scintillation Counting is an older technique that can be used to analyze the distribution of ¹⁴C in a compound; see, for example, Kent et al., “A Method for Obtaining the ¹⁴C-Isotope Distribution in Malate (C-2,3),” Anal. Biochem. 80, 176-182 (1977). More recently, accelerator mass spectrometry can be used to analyze the distribution of ¹⁴C in a compound. In the present invention, partially bio-based compounds can be used to study metabolic transformations, transport and/or distribution of medicines. This can be done by administering to a human or non-human subject, a fully bio-based, or, preferentially, a partially bio-based compound; then collecting the samples from within the body or excreted from the body. Typically, the samples will be concentrated (if necessary, collected from multiple subjects and concentrated) and analyzed for the presence, concentration and/or distribution of ¹⁴C. If desired, the results can be compared with a conventional, non-bio-based medicine having the same structure.

In some preferred embodiments of the present invention, aromatic starting materials are provided by the pyrolysis of biomass (preferably the pyrolysis of plant materials); for example, by the methods of Huber et al. incorporated herein. Thus, preferred starting materials include bio-based benzene, toluene and xylenes. Other aromatic starting materials such as naphthalene and thiophene may be used and are also derivable from the pyrolysis of biomass.

In an inventive aspect, a pharmaceutically effective dose of a bio-based or partially bio-based pharmaceutically active compound or pharmaceutical composition is provided. In some embodiments, the dose comprises a pharmaceutical composition comprising any one of the pharmaceutically active compounds shown in Table 1. The composition can be the pure active ingredient or can be a mixture with inert and/or other pharmacologically active compounds. The compound can be selected from any one of the compounds shown in Table 1. To provide one example, the pharmaceutically effective dose of a bio-based or partially bio-based lansoprazol molecule, depicted below.

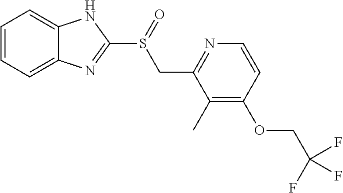 [SMILES: Cc1c(OCC(F)(F)F)ccnc1CS(=O)c1nc2ccccc2[nH]1]

This compound can be fully bio-based, or where only the phenyl group (not the pyridine group) is bio-based; or where at least 20%, at least 30%, at least 40%, at least 50%, at least 60%, at least 70%, at least 80%, or at least 90%, or from 30% to 90%, or from 30% to 80%, or from 40% to 90%, or from 50% to 100% of the carbon atoms in the lansoprazole structure are bio-based. The compound can be substantially completely bio-based. Each of compounds in Table 1, one at a time, replacing “lansoprazol” in the example above, is contemplated.

Thus, in another example, a pharmaceutically effective dose of a bio-based or partially bio-based cetirizine is provided, as depicted below:

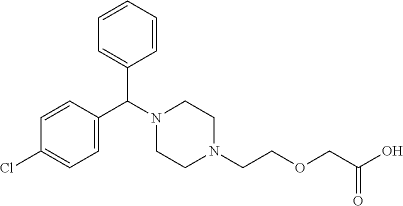 [SMILES: O=C(O)COCCN1CCN(C(c2ccccc2)c2ccc(Cl)cc2)CC1]

This compound can be fully bio-based, or where only the phenyl groups are bio-based; or where at least 20%, at least 30%, at least 40%, at least 50%, at least 60%, at least 70%, at least 80%, or at least 90%, or from 30% to 90%, or from 30% to 80%, or from 40% to 90%, or from 50% to 100% of the carbon atoms in the cetirizine structure are bio-based. The compound can be substantially completely bio-based. Each of compounds in Table 1, one at a time, replacing “cetirizine” in the example above, is contemplated.

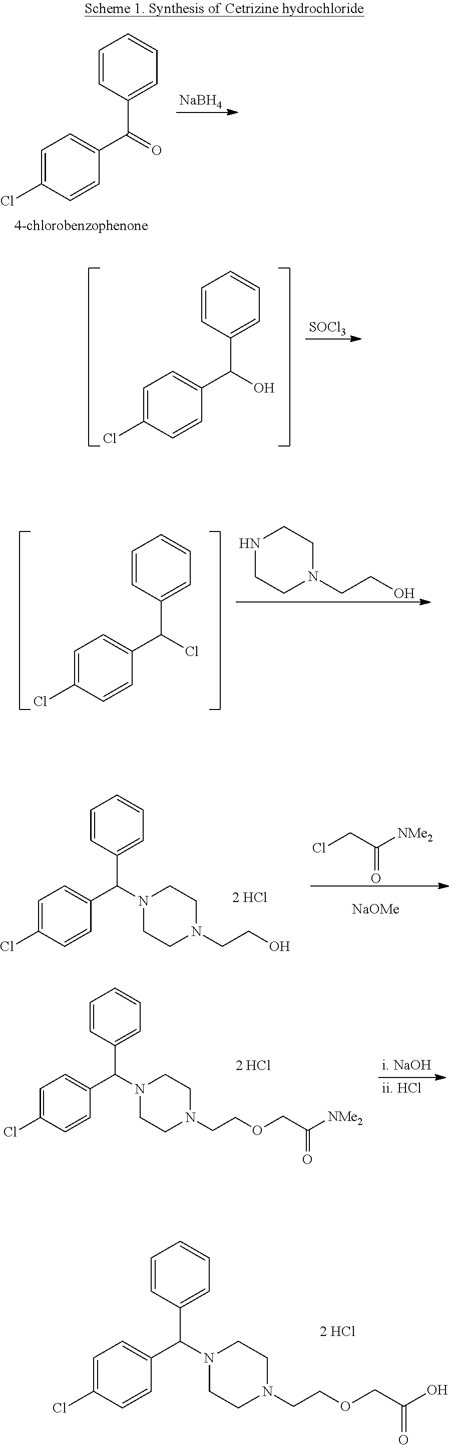

From the above synthesis of cetirizine, it can be readily seen that, in some preferred embodiments, the present invention provides cetirizine in which 12/20 of the carbon atoms (the carbon in the aryl groups) is bio-based, or 13/20 carbon atoms (including the tertiary carbon). Higher percentages can be provided via the use of non-aromatic bio-based compounds.

An alternative approach to citrizine dihydrochloride starts from 4-chlorobenzyl chloride (Guangdong Huagong, 2008, 35, 66-67) (Scheme 2).

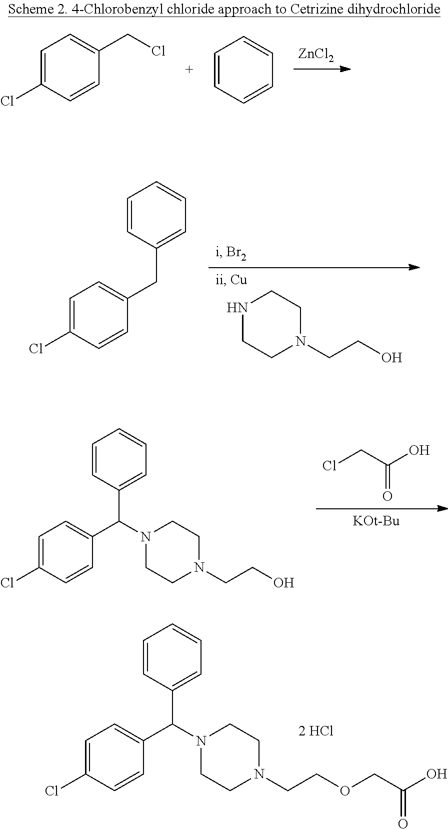

The most common approaches to cetirizine dihydrochloride utilize chemistry that incorporate 4-chlorobenzophenone or 4-chlorobenzhydrol. A selection of routes is shown below (Scheme 3)

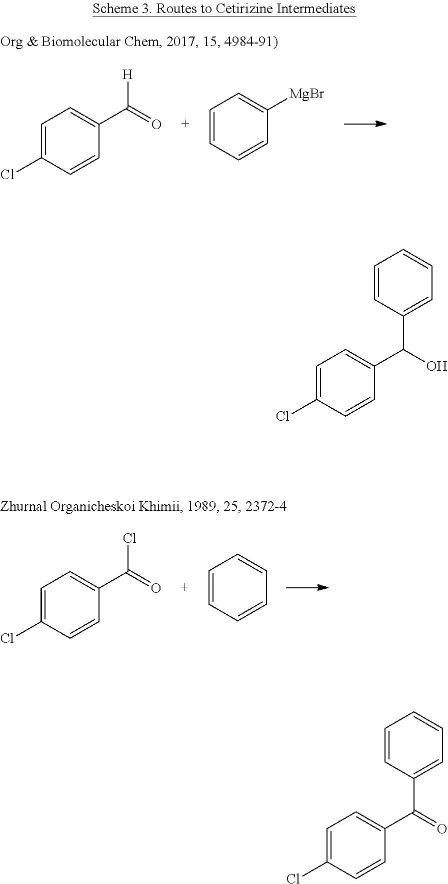

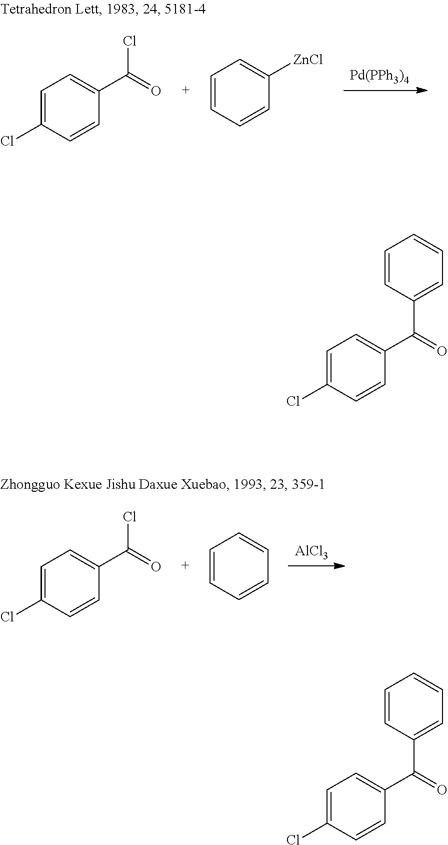

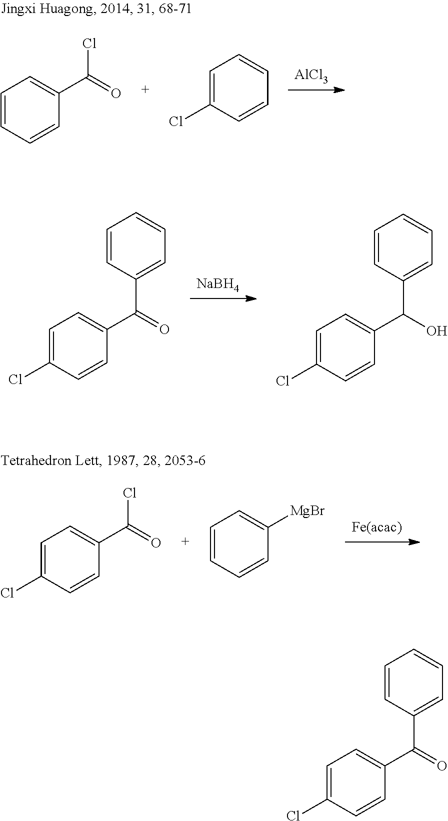

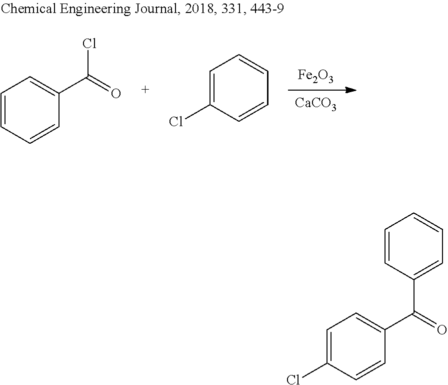

The basic chemicals used in the above chemistries could be sourced from benzene or toluene—examples are shown in Scheme 4.

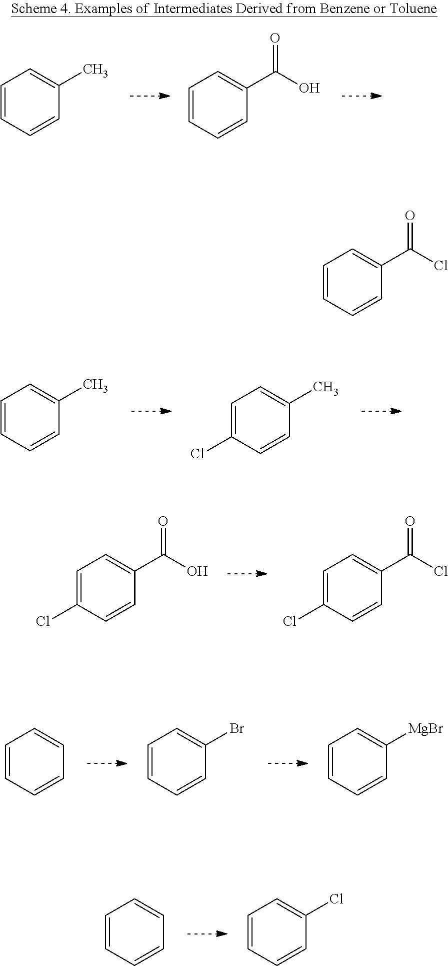

### Synthesis of Chlorhexidine (Chlohexamed Forte)

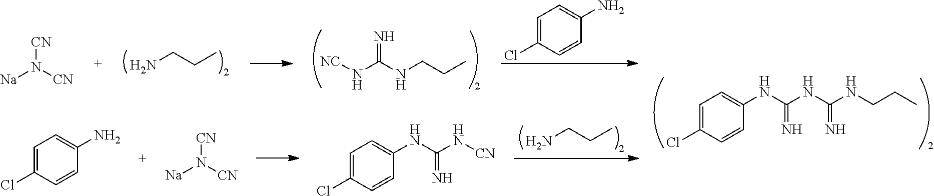

From this synthesis of chorhexidine it can be seen that, in some preferred embodiments, the present invention provides chlorhexidine in which 12/22 of the carbon atoms (the carbon in the aryl groups) is bio-based, or higher if bio-based alkyl amines are used.
Intermediate 4-Chloroaniline from Benzene:

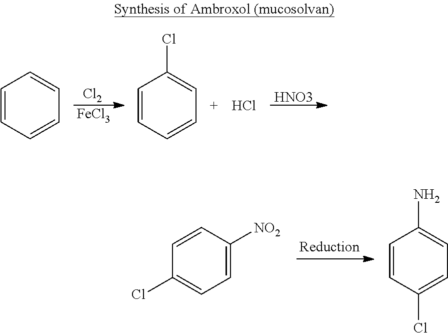

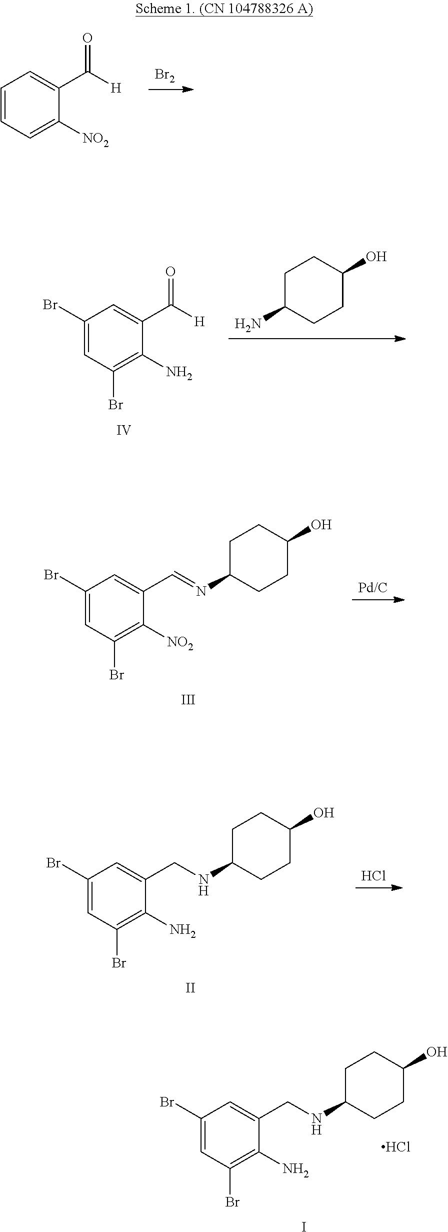

Intermediate 2-Nitrobenzaldehyde from Toluene:
Step 1: Gerald Booth (2007). “Nitro Compounds, Aromatic”. Ullmann's Encyclopedia of Industrial Chemistry. Weinheim: Wiley-VCH.
Step 2: Lauth, Bull. Soc. Chim. France, (3) 31, 133 (1904).

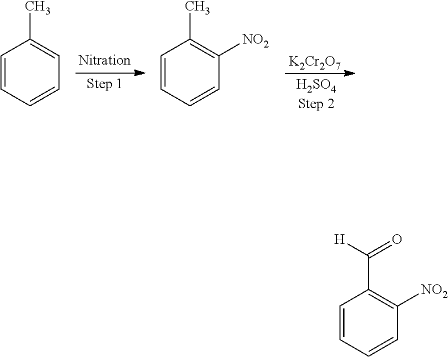

From the above synthesis of ambroxol, it can be seen that, in some preferred embodiments, the present invention provides ambroxol in which 7/13 of the carbon atoms (the carbon in the toluene group) is bio-based, or higher if bio-based nonaromatic starting materials are used.

### Synthesis of Bisacodyl (Ducolax)

Scheme 1. Kottler et al. U.S. Pat. No. 2,764,590 Certain 4, 4′-disubstituted-diphenylpyridyl methanes and process.

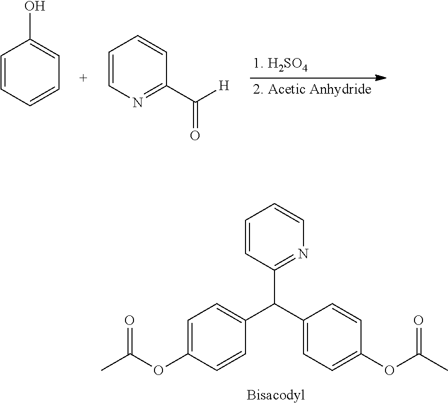 [SMILES: Oc1ccccc1 ; O=Cc1ccccn1 ; CC(=O)Oc1ccc(C(c2ccc(OC(C)=O)cc2)c2ccccn2)cc1]

From the above synthesis of bisacodyl, it can be seen that, in some preferred embodiments, the present invention provides chlorhexidine in which 12/22 of the carbon atoms (the carbon in the aryl groups) is bio-based, or 16/22 including bio-based acetic anhydride.
Intermediate Phenol from Toluene

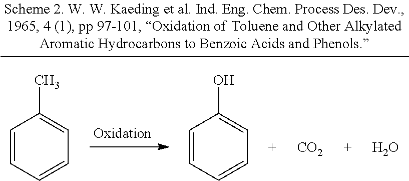

### Synthesis of Xylomethazoline (Olynth)

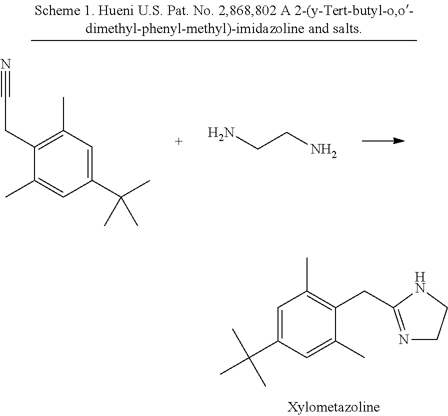

Intermediate Para-Tertiary-Butyl-Ortho:Ortho′-Dimethyl-Phenyl-Acetonitrile from m-Xylene.

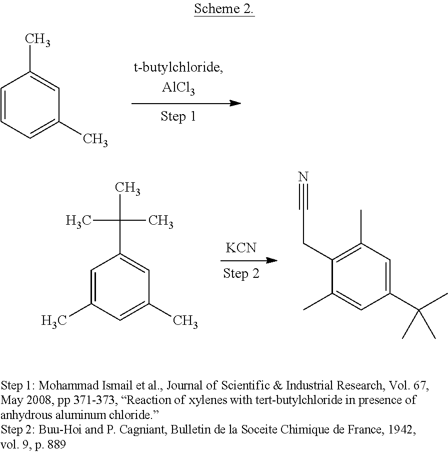

From the above synthesis of xylomethazoline, it can be seen that, in some preferred embodiments, the present invention provides xylomethazoline in which 8/14 of the carbon atoms (the carbon from xylene) is bio-based, or 10/14 including bio-based alkyl amine.

### Synthesis of Diclofenac (Forte Voltaren)

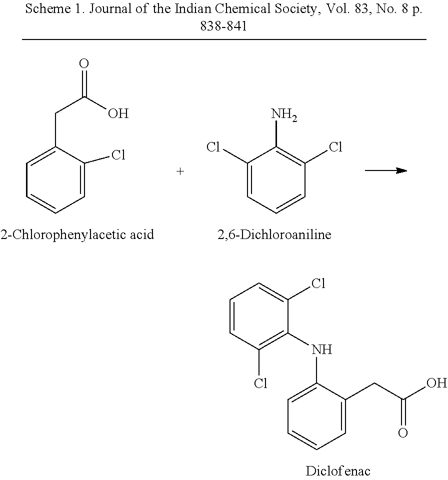 [SMILES: O=C(O)Cc1ccccc1Cl ; Nc1c(Cl)cccc1Cl ; O=C(O)Cc1ccccc1Nc1c(Cl)cccc1Cl]

From the synthesis of Diclofenac, it can be seen that, in some preferred embodiments, the present invention provides Diclofenac in which 13/14 of the carbon atoms (the carbon from benzene and toluene) is bio-based, or 14/14 including bio-based compound to result in the carboxylic acid group.

### Intermediates:

2,6-Dichloroaniline from Benzene

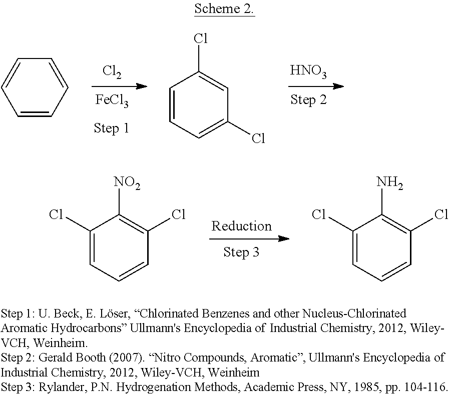

2-Chlorophenylacetic Acid from Toluene

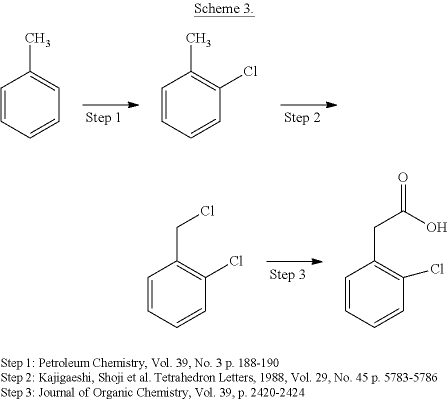

### Synthesis of Clotrimazole (Canesten)

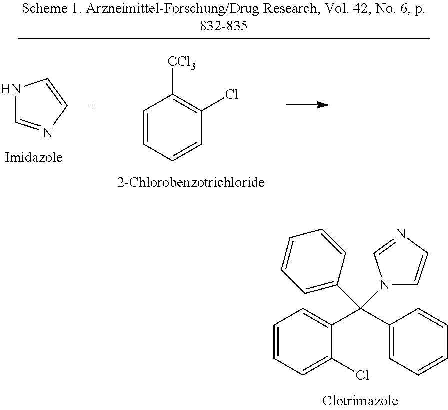

From the synthesis of Clotrimazole, it can be seen that, in some preferred embodiments, the present invention provides Clotrimazole in which 17/20 of the carbon atoms are bio-based.
Intermediate 2-Chlorobenzotrichloride from Toluene

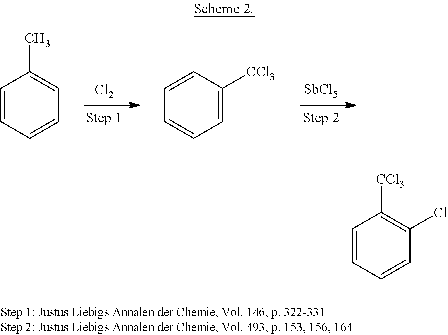

### Synthesis of Omeprazole (Omep Hexal)

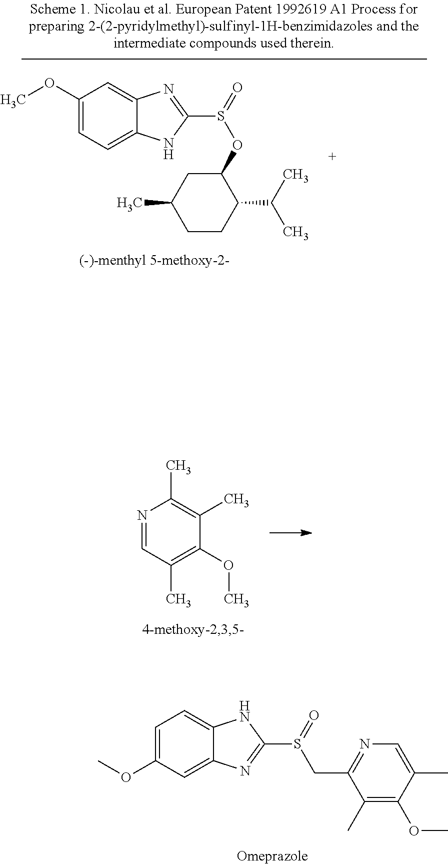

### Intermediates: 4-methoxy-2,3,5-trimethylpyridine from (Z)-3-Amino-2-methyl-2-butenoic Acid Ethyl Ester and Diethyl Methylmalonate

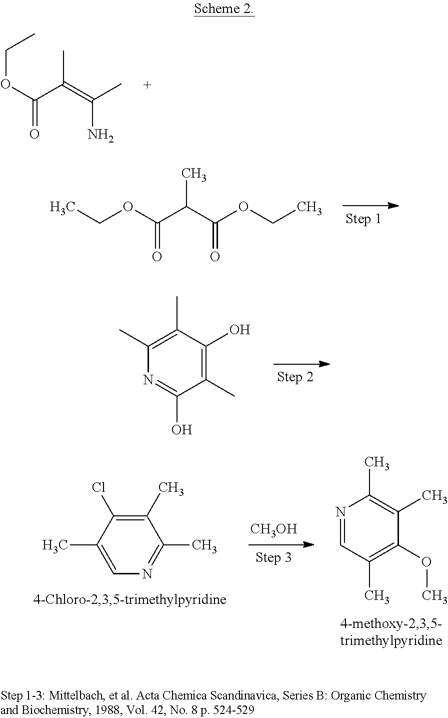

From the above synthesis of Omeprazole, it can be seen that, in some preferred embodiments, the present invention provides Omeprazole in which 6/17 of the carbon atoms are bio-based; higher concentrations of bio-based carbon atoms can be obtained from bio-based nonaromatic compounds.

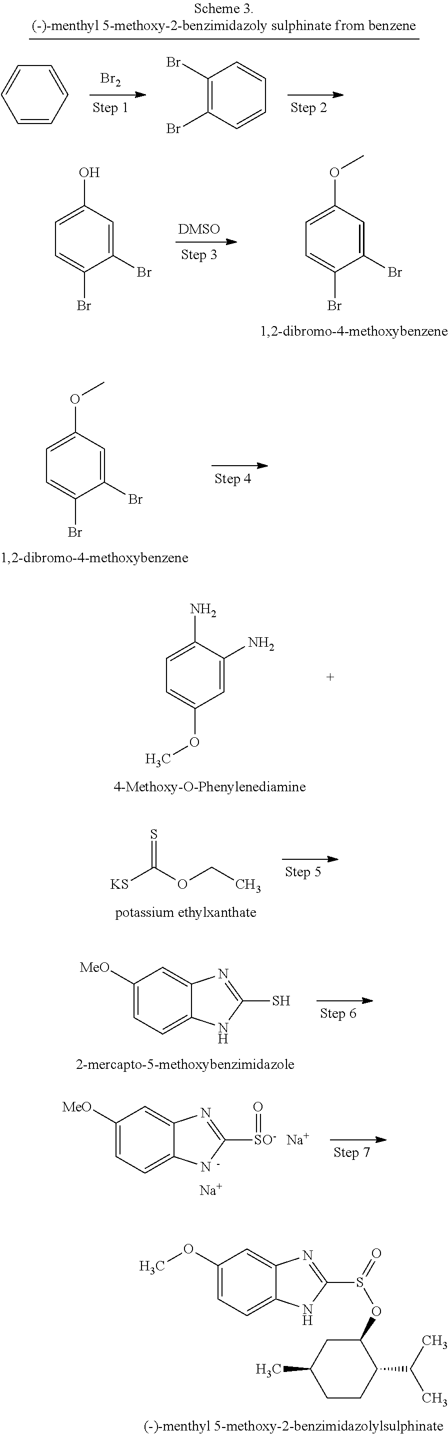

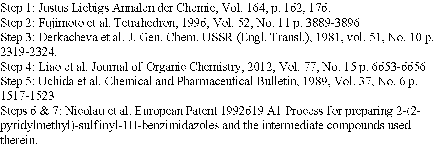

### Synthesis of Flurbiprofen (Dobendan)

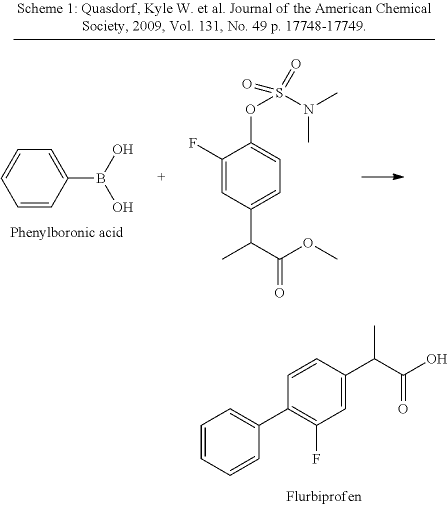 [SMILES: OB(O)c1ccccc1 ; COC(=O)C(C)c1ccc(OS(=O)(=O)N(C)C)c(F)c1 ; CC(C(=O)O)c1ccc(-c2ccccc2)c(F)c1]

From the above synthesis of Flurbiprofen, it can be seen that, in some preferred embodiments, the present invention provides Flurbiprofen in which 6/15 of the carbon atoms are bio-based (from the aryl group in phenylboronic acid); or 12/15 if both aryl groups are bio-based.

Intermediate Phenylboronic Acid from Benzene:

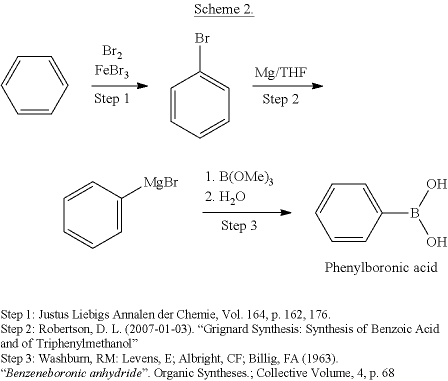

### Synthesis of Naproxen (Dolormin)

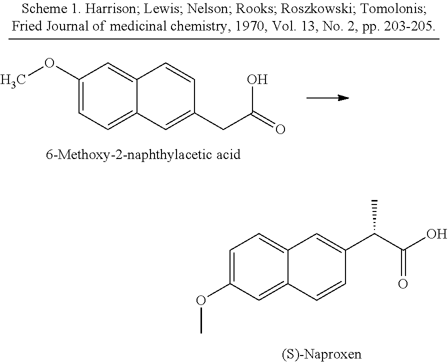 [SMILES: COc1ccc2cc(CC(=O)O)ccc2c1 ; COc1ccc2cc([C@H](C)C(=O)O)ccc2c1]

Intermediate 6-Methoxy-2-Naphthylacetic Acid from Naphthalene:

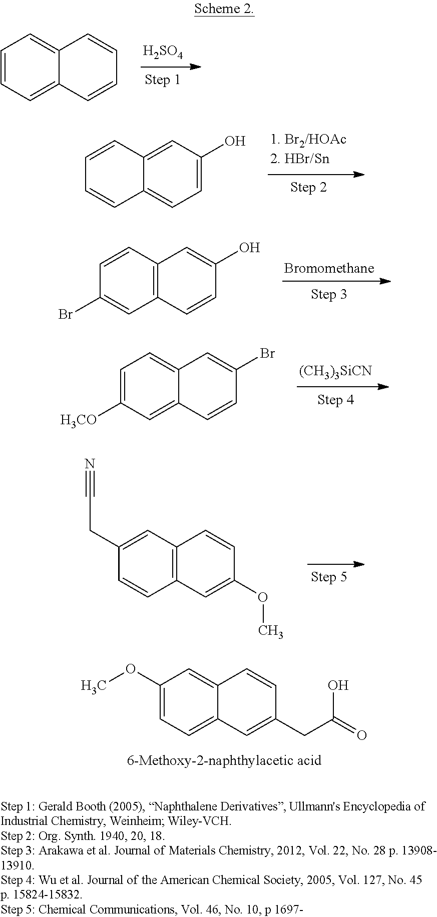

From the above synthesis of Naproxen, it can be seen that, in some preferred embodiments, the present invention provides Naproxen in which 10/14 of the carbon atoms are bio-based (from the naphthalene); or 11/14 or 14/14 via the use of bio-based reagents.

### Synthesis of Doxilamine (Hoggar)

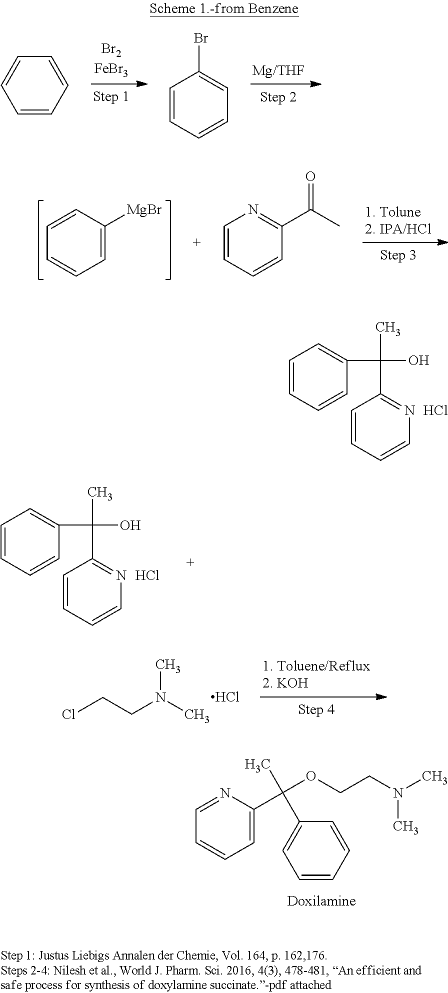

From the above synthesis of Doxilamine, it can be seen that, in some preferred embodiments, the present invention provides Doxilamine in which 6/17 of the carbon atoms are bio-based (from benzene); or 10/17 or more via the use of bio-based reagents.

### Synthesis of Ioperamide

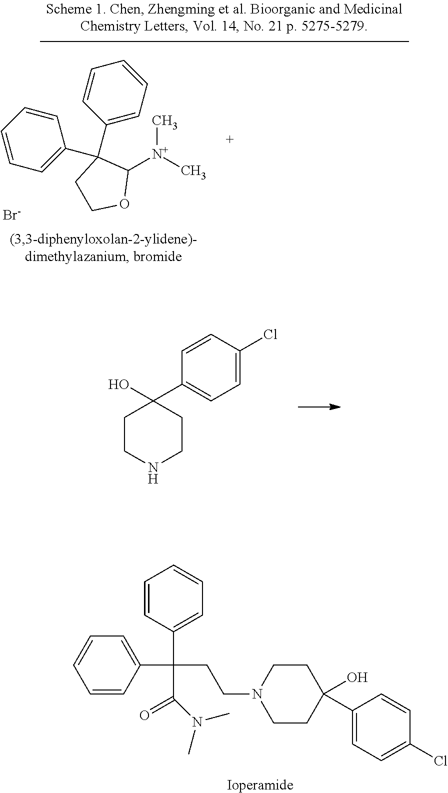

Intermediate (3,3-Diphenyloxolan-2-Ylidene)-Dimethylazanium,Bromide from Benzene
From the synthesis of Ioperamide, it can be seen that, in some preferred embodiments, the present invention provides Ioperamide in which 18/29 of the carbon atoms are bio-based (from aryl groups); or 16/29 or 22/29 (including bio-based ethyl acetate) or more via the use of bio-based reagents.

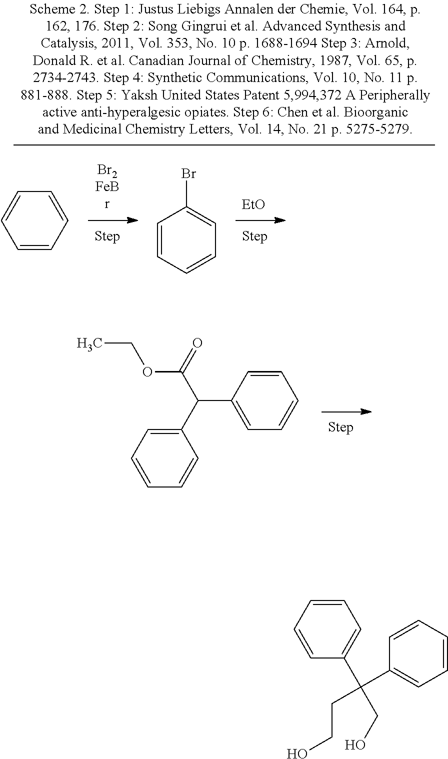 [SMILES: c1ccccc1 ; Brc1ccccc1 ; CCOC(=O)C(c1ccccc1)c1ccccc1 ; OCCC(CO)(c1ccccc1)c1ccccc1]

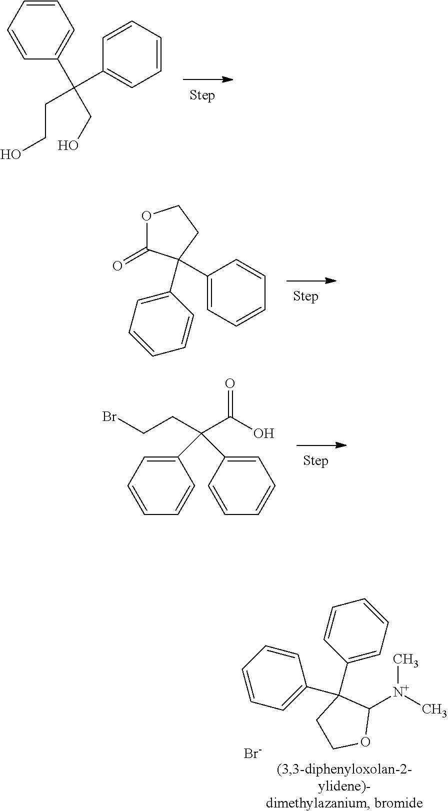

In another example, a pharmaceutically effective dose of a bio-based or partially bio-based mephentermine is disclosed, as depicted below.

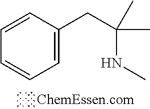

This compound can be fully bio-based, or where only the phenyl group is bio-based; or where at least 20%, at least 30%, at least 40%, at least 50%, at least 60%, at least 70%, at least 80%, or at least 90%, or from 30% to 90%, or from 30% to 80%, or from 40% to 90%, or from 50% to 100% of the carbon atoms in the mephentermine structure are bio-based. The compound can be substantially completely bio-based. Each of compounds in Table 1, one at a time, replacing “mephentermine” in the example above, is contemplated.

In another preferred embodiment, the active compound is ibuprofen:

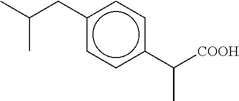

This compound can be fully bio-based, or where only the phenyl group is bio-based; or where at least 20%, at least 30%, at least 40%, at least 50%, at least 60%, at least 70%, at least 80%, or at least 90%, or from 30% to 90%, or from 30% to 80%, or from 40% to 90%, or from 50% to 100% of the carbon atoms in the ibuprofen structure are bio-based. The compound can be substantially completely bio-based.

In another embodiment, a method is disclosed of treating a patient comprising administering or prescribing a pharmaceutically effective dose of a bio-based, or partially bio-based, pharmaceutically active compound or pharmaceutical composition. In some embodiments, the patient is aware of or otherwise knows that the compound or composition is bio-based. In some cases, the method includes a step of informing the patient that the composition comprises a bio-based active ingredient. The patient can be informed verbally or in writing (such as via a label), or both.

In another embodiment, a method is disclosed of treating a patient wherein the bio-based pharmaceutically effective material is used in a treatment with another drug or drugs, either as a common dosage comprising both materials, or in a sequential treatment wherein the bio-based material and other material(s) are administered in a regimen that includes both materials.

Compositions that are disclosed can contain a conventional pharmaceutically active compound in addition to a bio-based pharmaceutically active compound.

This disclosure is not limited to any particular method or methods by which the pharmaceutically active compound are made. Typically, the inventive structures are made using products obtained by pyrolyzing biomass in the presence of a catalyst. The catalyzed pyrolysis process can be conducted to produce high yields of aromatics, especially benzene, toluene, and xylenes. The subsequent use of these bio-based aromatics in the synthesis of drug structures can produce drug structures in which the aromatic rings (optionally with attached methyl or methoxy groups) are bio-based.

The partially or fully bio-based compounds and compositions described herein replace conventional pharmaceutical compounds and compositions that are derived from petro-chemicals. Most “natural products” are merely identified based on their presence in nature, but are prepared via petrochemical-based synthetic chemical processes at a commercial-scale. The rare commercially-available pharmaceutically active compound that is prepared via fermentation process or via extraction from a natural source would be “bio-sourced” (and have the telltale isotopic ¹⁴C/¹²C ratio) and these commercially-available pharmaceutically active compound are not included in the subject matter being claimed; although with respect to the commercially-available pharmaceutically active compounds that are only partially bio-sourced; partially or fully bio-based compounds and compositions that have a higher mass % of bio-based carbon are included in the subject matter being claimed.

In some preferred embodiments, the drug structure is cetirizine or other antihistamine that contains an aromatic ring structure. In some preferred embodiments, the drug structure is produced using at least in part bio-based benzene, toluene, or xylene, or C9+ aromatics or some mixture of these. In some embodiments, the pharmaceutically effective dose is in the form of a tablet, capsule, injectable or other dosage form having a mass of drug of at least 0.1 mg, or at least 0.5 mg, or at least 1 mg, or at least 5 mg or at least 10 mg, or from 0.01 to 10 mg, or from 0.5 to 5 mg.

### Examples—Patient Compliance

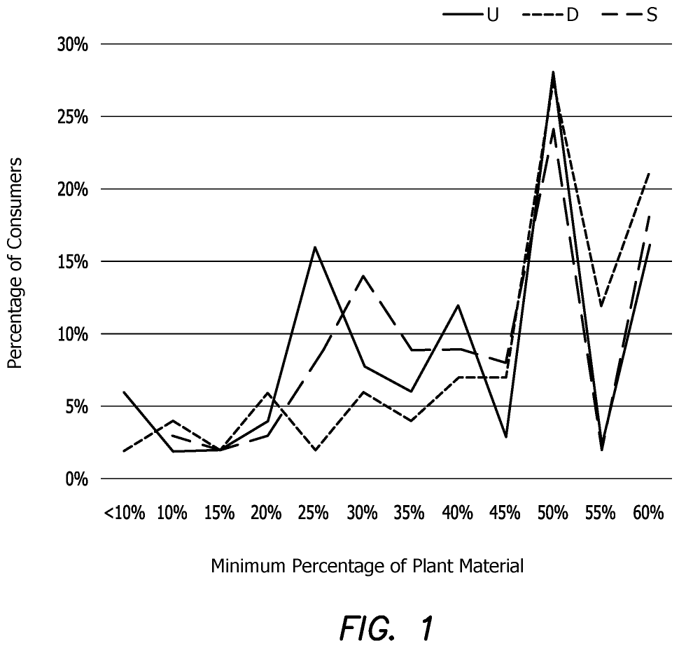

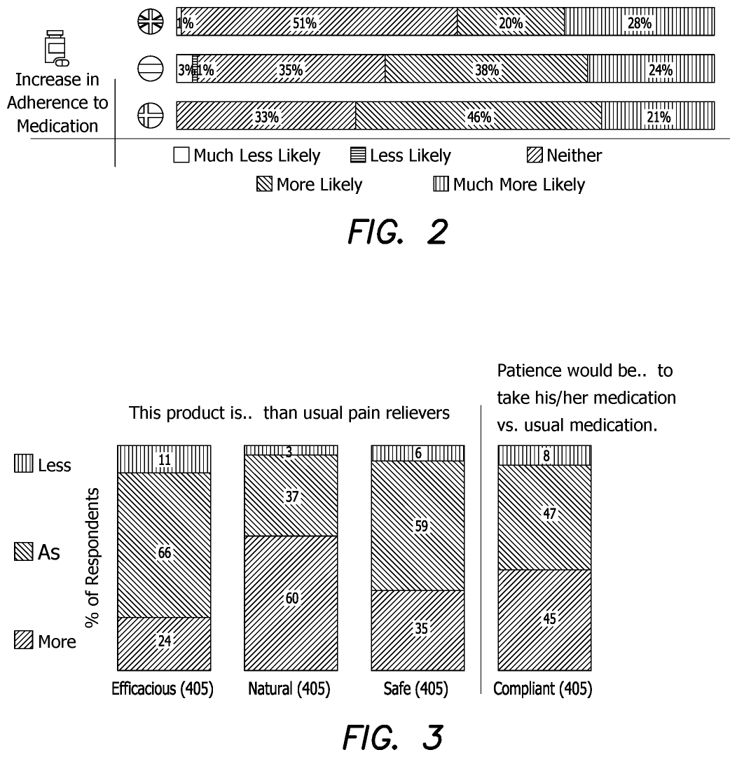

A survey was conducted of 101 residents of the United Kingdom (UK), 106 German (DE) residents, and 63 Swedish (SE) residents, all of whom use Cetirizine. The people in this survey were asked a series of questions about Cetirizine. As can be seen in FIG. 1, a higher percentage of plant material in the Cetirizine would lead more people to purchase the bio-based medicine. The people in the survey were also asked “Do you think you would be more likely to finish a complete course of your medication if it was manufactured from environmentally-friendly renewable raw plant materials than the same medication that was made from standard materials/synthetic chemicals? Please rank on a scale of 1-5 where 1 is much less likely and 5 is much more likely.” From this question, it was discovered, very surprisingly, that bio-based Cetirizine, would increase compliance (adherence), making people between 4800 and 67% more likely or much more likely to comply with taking the medication. These results are shown in FIG. 2.

A similar result was obtained for the drug ibuprofen. After being shown packaging for ibuprofen made with 50% plant raw materials, respondents were asked whether a “patient would more likely, as likely, or less likely . . . to be compliant to take his/her medication versus usual medication.” As can be seen in FIG. 3, a survey of 405 German residents surprisingly showed that 45 of respondents thought that patients would be more compliant in taking the bio-based medication.

Thus, the data shows that the use of bio-based medicines (which possess an elevated ¹⁴C/¹²C ratio relative to fossil fuels) lead to surprisingly improved levels of patient compliance.

TABLE 1

| | Molecular formula |
|---|---|
| foscarnet; Phosphonoformic acid; Phosphonoformate . . . | CH3O5P |
| Clodronate; Clodronic acid; Dichloromethanediphosphonic acid . . . | CH4Cl2O6P2 |
| Etidronic acid; etidronate; HEDP . . . | C2H8O7P2 |
| halothane; Fluothane; Narcotan . . . | C2HBrClF3 |
| pamidronate; PAMIDRONIC acid; Amidronate . . . | C3H11NO7P2 |
| enflurane; Ethrane; Methylflurether . . . | C3H2ClF5O |
| isoflurane; Forane; Aerrane . . . | C3H2ClF5O |
| Suprane; DESFLURANE; Desfluranum [INN-Latin] . . . | C3H2F6O |
| methoxyflurane; Penthrane; Anecotan . . . | C3H4Cl2F2O |
| nitroglycerin; Nitrostat; Glyceryl trinitrate . . . | C3H5N3O9 |
| FOSFOMYCIN; phosphomycin; Phosphonomycin . . . | C3H7O4P |
| fosmidomycin; Fosmidomycina; Fosmidomycine . . . | C4H10NO5P |
| metformin; Glucophage; Glumetza . . . | C4H11N5 |
| TROMETHAMINE; Trometamol; Tris . . . | C4H11NO3 |
| alendronate; ALENDRONIC acid; Fosamax . . . | C4H13NO7P2 |
| sevoflurane; Ultane; Sevofluran . . . | C4H3F7O |
| 5-Fluorouracil; fluorouracil; 5-FU . . . | C4H3FN2O2 |
| methimazole; thiamazole; Tapazole . . . | C4H6N2S |
| acetazolamide; Diamox; Diacarb . . . | C4H6N4O3S2 |
| Zoledronic acid; Zoledronate; Reclast . . . | C5H10N2O7P2 |
| mechlorethamine; Chlormethine; Nitrogen mustard . . . | C5H11Cl2N |
| D-Penicillamine; Cuprimine; Depen . . . | C5H11NO2S |
| Acamprosate; N-Acetylhomotaurine; Campral . . . | C5H11NO4S |
| Orotic acid; 6-Carboxyuracil; Orodin . . . | C5H4N2O4 |
| allopurinol; Zyloprim; Lopurin . . . | C5H4N4O |
| 6-Mercaptopurine; mercaptopurine; Purinethol . . . | C5H4N4S |
| pyrazinamide; pyrazinecarboxamide; pyrazinoic acid amide . . . | C5H5N3O |
| 6-Thioguanine; thioguanine; Tioguanine . . . | C5H5N5S |
| methazolamide; Methenamide; Neptazaneat . . . | C5H8N4O3S2 |
| carmustine; BCNU; Carmustin . . . | C5H9Cl2N3O2 |
| histamine; 1H-Imidazole-4-ethanamine; 2-(4-Imidazolyl)ethylamine . . . | C5H9N3 |
| BETAZOLE; Ametazole; 1H-Pyrazole-3-ethanamine . . . | C5H9N3 |
| L-proline; proline; L-(−)-Proline . . . | C5H9NO2 |
| N-Acetyl-L-cysteine; acetylcysteine; N-Acetylcysteine . . . | C5H9NO3S |
| L-glutamic acid; L-glutamate; glutacid . . . | C5H9NO4 |
| MEGLUTOL; 3-Hydroxy-3-methylglutaric acid; Dicrotalic acid . . . | C6H10O5 |
| vigabatrin; Sabril; gamma-Vinyl GABA . . . | C6H11NO2 |
| 6-aminohexanoic acid; aminocaproic acid; 6-Aminocaproic acid . . . | C6H13NO2 |
| Isofagomine; Afegostat; (3R,4R,5R)-5-(HYDROXYMETHYL)PIPERIDINE- | C6H13NO3 |
| 3,4-DIOL . . . | |
| L-lysine; lysine; lysine acid . . . | C6H14N2O2 |
| L-arginine; arginine; L-(+)-Arginine . . . | C6H14N4O2 |
| busulfan; Myleran; Busulphan . . . | C6H14O6S2 |
| Carbacholin; Carbacholine; Carbacolina . . . | C6H15N2O2+ |
| dichlorphenamide; Diclofenamide; Dichlofenamide . . . | C6H6Cl2N2O4S2 |
| lindane; Kwell; beta-BHC . . . | C6H6Cl6 |
| nicotinamide; niaClnamide; 3-Pyridinecarboxamide . . . | C6H6N2O |
| temozolomide; Methazolastone; Temodar . . . | C6H6N6O2 |
| isoniazid; Isonicotinic acid hydrazide; Isonicotinohydrazide . . . | C6H7N3O |
| AMILORIDE; Amipramidin; Midamor . . . | C6H8ClN7O |
| sulfanilamide; 4-Aminobenzenesulfonamide; Sulphanilamide . . . | C6H8N2O2S |
| ascorbic acid; I-ascorbic acid; vitamin C . . . | C6H8O6 |
| citric acid; Citro; citrate . . . | C6H8O7 |
| metronidazole; Flagyl; Metronidazol . . . | C6H9N3O3 |
| trimethadione; Troxidone; Tridione . . . | C6H9NO3 |
| ISOSORBIDE MONONITRATE; Monoket; Imdur . . . | C6H9NO6 |
| Carbimazole; Athyromazole; Carbimazol . . . | C7H10N2O2S |
| propylthiouracil; 6-Propyl-2-thiouracil; Procasil . . . | C7H10N2OS |
| ethosuximide; Zarontin; Etosuximida . . . | C7H11NO2 |
| PARAMETHADIONE; Paradione; Isoethadione . . . | C7H11NO3 |
| Risedronate; Risedronic acid; 105462-24-6 . . . | C7H11NO7P2 |
| cyclophosphamide; Cyclophosphamid; Procytox . . . | C7H15Cl2N2O2P |
| ifosfamide; Isophosphamide; Iphosphamide . . . | C7H15Cl2N2O2P |
| L-carnitine; Levocarnitine; Carnitor . . . | C7H15NO3 |
| chlorzoxazone; Paraflex; Biomioran . . . | C7H4ClNO2 |
| chlorothiazide; Diuril; Chlotride . . . | C7H6ClN3O4S2 |
| salicylic acid; 2-Hydroxybenzoic acid; o-hydroxybenzoic acid . . . | C7H6O3 |
| 5-Aminosalicylic acid; mesalamine; Mesalazine . . . | C7H7NO3 |
| hydrochlorothiazide; Hypothiazide; Esidrix . . . | C7H8ClN3O4S2 |
| ethchlorvynol; Ethchlorovynol; Placidyl . . . | C7H9ClO |
| Emtricitabine; Coviracil; Emtriva . . . | C8H10FN3O3S |
| sulfacetamide; Sulphacetamide; Acetosulfamine . . . | C8H10N2O3S |
| ethionamide; Trecator; Ethioniamide . . . | C8H10N2S |
| ENPROFYLLINE; 3-Propylxanthine; 3-n-Propylxanthine . . . | C8H10N4O2 |
| lamivudine; Epivir; Zeffix . . . | C8H11N3O3S |
| acyclovir; Acycloguanosine; Aciclovir . . . | C8H11N5O3 |
| dopamine; Hydroxytyramin; intropin . . . | C8H11NO2 |
| pyridoxine; Pyridoxol; Gravidox . . . | C8H11NO3 |
| norepinephrine; noradrenaline; Arterenol . . . | C8H11NO3 |
| betahistine; 2-(2-METHYLAMINOETHYL)PYRIDINE; Vasomotal . . . | C8H12N2 |
| Phenelzine; Phenethylhydrazine; Nardil . . . | C8H12N2 |
| barbital; Barbitone; Veronal . . . | C8H12N2O3 |
| Decitabine; 5-Aza-2′-deoxycytidine; Dacogen . . . | C8H12N4O4 |
| ribavirin; Tribavirin; Virazole . . . | C8H12N4O5 |
| 5-azacytidine; Azacitidine; Azacytidine . . . | C8H12N4O5 |
| LEVETIRACETAM; Levetiracetam [INN]; Levetiracetamum [INN-Latin] | C8H14N2O2 |
| famotidine; PEPCID; Famodil . . . | C8H15N7O2S3 |
| VALPROIC acid; 2-Propylpentanoic acid; Depakene . . . | C8H16O2 |
| Pregabalin; Lyrica; 3-isobutyl GABA . . . | C8H17NO2 |
| MIGLITOL; Glyset; 72432-03-2 . . . | C8H17NO5 |
| riluzole; Rilutek; 1744-22-5 . . . | C8H5F3N2OS |
| diazoxide; Proglycem; Eudemine . . . | C8H7ClN2O2S |
| furazolidone; Furoxone; Furazolidine . . . | C8H7N3O5 |
| guanabenz; Wy 8678 base; Wytensin . . . | C8H8Cl2N4 |
| trichlormethiazide; Trichloromethiazide; Naqua . . . | C8H8Cl3N3O4S2 |
| hydroflumethiazide; Diuredemina; Diurometon . . . | C8H8F3N3O4S2 |
| zonisamide; Zonegran; Exceglan . . . | C8H8N2O3S |
| hydralazine; 1-Hydrazinophthalazine; Apresolin . . . | C8H8N4 |
| acetaminophen; Paracetamol; 4-Acetamidophenol . . . | C8H9NO2 |
| CLAVULANIC acid; Clavulanate; Antibiotic MM 14151 . . . | C8H9NO5 |
| Bivalirudin; Angiomax; Hirulog . . . | C98H138N24O33 |
| apraclonidine; lopidine; 4-Aminoclonidine . . . | C9H10Cl2N4 |
| phenacemide; Phenuron; Phenurone . . . | C9H10N2O2 |
| aminohippuric acid; 4-Aminohippuric acid; P-AMINOHIPPURIC acid . . . | C9H10N2O3 |
| sulfamethizole; Sulphamethizole; Sulfamethizol . . . | C9H10N4O2S2 |
| methyclothiazide; Aquatensen; Enduron . . . | C9H11Cl2N3O4S2 |
| gemcitabine; dFdC; Gemcin . . . | C9H11F2N3O4 |
| floxuridine; 2′-Deoxy-5-fluorouridine; FUDR . . . | C9H11FN2O5 |
| idoxuridine; 5-lodo-2′-deoxyuridine; Stoxil . . . | C9H11IN2O5 |
| benzocaine; Ethyl 4-aminobenzoate; Americaine . . . | C9H11NO2 |
| L-phenylalanine; phenylalanine; 3-Phenyl-L-alanine . . . | C9H11NO2 |
| L-tyrosine; tyrosine; (S)-Tyrosine . . . | C9H11NO3 |
| levodopa; L-dopa; Dopar . . . | C9H11NO4 |
| dextroamphetamine; Dexamphetamine; D-Amphetamine . . . | C9H13N |
| iproniazid; Marsilid; Rivivol . . . | C9H13N3O |
| zalcitabine; Dideoxycytidine; 2′,3′-DIDEOXYCYTIDINE . . . | C9H13N3O3 |
| cytarabine; Ara-C; Cytosine arabinoside . . . | C9H13N3O5 |
| ganciclovir; Cytovene; Gancyclovir . . . | C9H13N5O4 |
| phenylpropanolamine; Norephedrine; Rhindecon . . . | C9H13NO |
| ethinamate; Ethinamat; Valmid . . . | C9H13NO2 |
| metaraminol; L-Metaraminol; Hydroxynorephedrine . . . | C9H13NO2 |
| phenylephrine; Neosynephrine; Mezaton . . . | C9H13NO2 |
| levonordefrin; Nordefrin; Neo-Cobefrin . . . | C9H13NO3 |
| epinephrine; Adrenalin; I-Adrenaline . . . | C9H13NO3 |
| metharbital; Gemonil; Methylbarbital . . . | C9H14N2O3 |
| minoxidil; Loniten; Rogaine . . . | C9H15N5O |
| Kuvan; 5,6,7,8-tetrahydrobiopterin; tetrahydrobiopterin . . . | C9H15N5O3 |
| captopril; Capoten; Cesplon . . . | C9H15NO3S |
| lomustine; Belustine; CeeNU . . . | C9H16ClN3O2 |
| azelaic acid; NONANEDIOIC acid; Finacea . . . | C9H16O4 |
| gabapentin; Neurontin; Gabapentine . . . | C9H17NO2 |
| meprobamate; Amepromat; Equanil . . . | C9H18N2O4 |
| altretamine; Hexalen; Hexastat . . . | C9H18N6 |
| Ibandronic acid; Ibandronate; Bondronat . . . | C9H23NO7P2 |
| haloprogin; Halotex; Mycanden . . . | C9H4Cl3IO |
| chloroxine; 5,7-Dichloro-8-hydroxyquinoline; Capitrol . . . | C9H5Cl2NO |
| clioquinol; lodochlorhydroxyquin; Vioform . . . | C9H5ClINO |
| lamotrigine; Lamictal; Lamictal Cd . . . | C9H7Cl2N5 |
| azathioprine; Imuran; Azothioprine . . . | C9H7N7O2S |
| tizanidine; Sirdalud; Ternelin . . . | C9H8ClN5S |
| clonidine; Duraclon; Chlornidinum . . . | C9H9Cl2N3 |
| GUANFACINE; Estulic; Intuniv . . . | C9H9Cl2N3O |
| sulfadiazine; Sulphadiazine; Sulfapyrimidine . . . | C10H10N402S |
| Clofarabine; Clolar; Evoltra . . . | C10H11ClFN5O3 |
| Trifluridine; Viroptic; TRIFLUOROTHYMIDINE . . . | C10H11F3N2O5 |
| sulfamethoxazole; Sulfisomezole; Gantanol . . . | C10H11N3O3S |
| QUINETHAZONE; Chinethazonum; Quinethazon . . . | C10H12ClN3O3S |
| Cladribine; 2-Chloro-2′-deoxyadenosine; Leustatin . . . | C10H12ClN5O3 |
| baclofen; Lioresal; Baclon . . . | C10H12ClNO2 |
| tolazoline; Priscoline; 2-Benzylimidazoline . . . | C10H12N2 |
| stavudine; sanilvudine; Zerit . . . | C10H12N2O4 |
| didanosine; DIDEOXYINOSINE; Videx . . . | C10H12N4O3 |
| Tazobactam; 89786-04-9; CHEMBL404 . . . | C10H12N4O5S |
| chlorpropamide; Chloropropamide; Diabinese . . . | C10H13ClN2O3S |
| fludarabine phosphate; fludara; FAMP . . . | C10H13FN5O7P |
| Debrisoquine; Debrisoquin; Debrisochinum . . . | C10H13N3 |
| Vidarabine; Ara-A; Vira-A . . . | C10H13N5O4 |
| zidovudine; Azidothymidine; Retrovir . . . | C10H13N5O4 |
| adenosine; Adenocard; Adenoscan . . . | C10H13N5O4 |
| Tenamfetamine; 3,4-methylenedioxyamphetamine; | C10H13NO2 |
| Methylenedioxyamphetamine . . . | |
| phenacetin; Acetophenetidin; Acetophenetin . . . | C10H13NO2 |
| Metirosine; METYROSINE; Methyltyrosine . . . | C10H13NO3 |
| methyldopa; Aldomet; Alphamethyldopa . . . | C10H13NO4 |
| Black leaf; L-Nicotine; Nicocide . . . | C10H14N2 |
| APROBARBITAL; Alurate; Allypropymal . . . | C10H14N2O3 |
| carbidopa; Lodosyn; N-Aminomethyldopa . . . | C10H14N2O4 |
| Telbivudine; Tyzeka; L-Thymidine . . . | C10H14N2O5 |
| dyphylline; Diprophylline; Glyphylline . . . | C10H14N4O4 |
| 5′-adenylic acid; adenylic acid; Adenosine monophosphate . . . | C10H14N5O7P |
| METHAMPHETAMINE; Metamfetamine; d-Deoxyephedrine . . . | C10H15N |
| phentermine; Duromine; Ionamin . . . | C10H15N |
| PHENFORMIN; Phenformine; Fenformin . . . | C10H15N5 |
| penciclovir; Denavir; Vectavir . . . | C10H15N5O3 |
| PSEUDOEPHEDRINE; Isoephedrine; d-Pseudoephedrine . . . | C10H15NO |
| pipobroman; Vercyte; Amedel . . . | C10H16Br2N2O2 |
| butabarbital; Secbutabarbital; Butisol . . . | C10H16N2O3 |
| dorzolamide; 1cil; Trusopt (TN) . . . | C10H16N2O4S3 |
| EDTA; Edetic acid; Ethylenediaminetetraacetic acid . . . | C10H16N2O8 |
| cimetidine; Tagamet; Cimetag . . . | C10H16N6S |
| amantadine; 1-Adamantanamine; 1-Aminoadamantane . . . | C10H17N |
| Dexpramipexole; 104632-28-2; Pramipexol . . . | C10H17N3S |
| methyprylon; Noludar; Metiprilon . . . | C10H17NO2 |
| GUANADREL; Hylorel; Guanadrelum [INN-Latin] . . . | C10H19N3O2 |
| disulfiram; Tetraethylthiuram disulfide; Antabuse . . . | C10H20N2S4 |
| I-Menthol; (−)-menthol; Levomenthol . . . | C10H20O |
| diethylcarbamazine; Carbamazine; Hetrazan . . . | C10H21N3O |
| Miglustat; Zavesca; N-Butylmoranoline . . . | C10H21NO4 |
| voglibose; Basen; Glustat . . . | C10H21NO7 |
| GUANETHIDINE; Ismelin; Oktadin . . . | C10H22N4 |
| anagrelide; Anagrelida; Anagrelidum . . . | C10H7Cl2N3O |
| thiabendazole; Tiabendazole; Mintezol . . . | C10H7N3S |
| Pemirolast; Pemirolastum [INN-Latin]; Pemirolast [INN] . . . | C10H8N6O |
| Amrinone; inamrinone; Inocor . . . | C10H9N3O |
| brimonidine; Bromoxidine; 59803-98-4 . . . | C11H10BrN5 |
| METOPRINE; Methodichlorophen; DDMP . . . | C11H10Cl2N4 |
| flutamide; Eulexin; Niftolide . . . | C11H11F3N2O3 |
| sulfapyridine; Sulphapyridine; 2-Sulfapyridine . . . | C11H11N3O2S |
| Lofexidine; Britlofex; Lofexidinum [INN-Latin] . . . | C11H12Cl2N2O |
| chlormezanone; Chlormethazanone; Trancopal . . . | C11H12ClNO3S |
| antipyrine; Phenazone; Anodynin . . . | C11H12N2O |
| ethotoin; Peganone; Ethotoine [INN-French] . . . | C11H12N2O2 |
| L-tryptophan; tryptophan; Tryptophane . . . | C11H12N2O2 |
| ZILEUTON; Zyflo; Leutrol . . . | C11H12N2O2S |
| levamisole; L-Tetramisole; Lepuron . . . | C11H12N2S |
| sulfamerazine; Sulphamerazine; Sulfamethyldiazine . . . | C11H12N4O2S |
| SULFALENE; Sulfamethopyrazine; Kelfizina . . . | C11H12N4O3S |
| polythiazide; Renese; Drenusil . . . | C11H13ClF3N3O4S3 |
| pargyline; Eudatin; Paragyline . . . | C11H13N |
| sulfisoxazole; Sulfafurazole; Sulphafurazole . . . | C11H13N3O3S |
| felbamate; Felbatol; Felbamyl . . . | C11H14N2O4 |
| Nelarabine; Nelzarabine; Arranon . . . | C11H15N5O5 |
| phenmetrazine; 3-Methyl-2-phenylmorpholine; Oxazimedrine . . . | C11H15NO |
| Brolamfetamine; 4-Bromo-2,5-dimethoxyamphetamine; DOB-4 . . . | C11H16BrNO2 |
| proguanil; Chloroguanide; Chlorguanide . . . | C11H16ClN5 |
| tocainide; Tonocard; Tocainidum [INN-Latin] . . . | C11H16N2O |
| pilocarpine; Isoptocarpine; Pilocarpin . . . | C11H16N2O2 |
| butalbital; Allylbarbital; Itobarbital . . . | C11H16N2O3 |
| Dexrazoxane; Zinecard; Cardioxane . . . | C11H16N4O4 |
| Bretylium; Bretylium tolsylate; Bretylum . . . | C11H17BrN+ |
| MEPHENTERMINE; Mephenterdrine; Mefenterdrin . . . | C11H17N |
| Mexiletine; Mexiletene; Mexiletina . . . | C11H17NO |
| isoproterenol; Isoprenaline; Norisodrine . . . | C11H17NO3 |
| metaproterenol; ORCIPRENALINE; Alupent . . . | C11H17NO3 |
| methoxamine; Methoxamedrine; Methoxamin . . . | C11H17NO3 |
| amobarbital; Amylobarbitone; Amytal . . . | C11H18N2O3 |
| pentobarbital; Pentobarbitone; Nembutal . . . | C11H18N2O3 |
| mecamylamine; Versamine; Mecamine . . . | C11H21N |
| menadione; Vitamin K3; 2-Methyl-1,4-naphthoquinone . . . | C11H8O2 |
| Sorbinil; Sorbinilo; Sorbinilum . . . | C11H9FN2O3 |
| nilutamide; Anandron; Nilandron . . . | C12H10F3N3O4 |
| cinoxacin; Cinobac; Compound 64716 . . . | C12H10N2O5 |
| furosemide; Frusemide; Lasix . . . | C12H11ClN2O5S |
| PELDESINE; Peldesine [USAN]; BCX-34 . . . | C12H11N5O |
| triamterene; Dyrenium; Noridil . . . | C12H11N7 |
| dapsone; Diaphenylsulfone; Sulfona . . . | C12H12N2O2S |
| ENOXIMONE; Perfan; Fenoximone . . . | C12H12N2O2S |
| nalidixic acid; Nalidixin; Nevigramon . . . | C12H12N2O3 |
| phenobarbital; Luminal; Phenobarbitol . . . | C12H12N2O3 |
| DAZOXIBEN; Dazoxiben [INN-Spanish]; Dazoxibene [INN-French] . . . | C12H12N2O3 |
| pyrimethamine; Daraprim; Ethylpyrimidine . . . | C12H13ClN4 |
| rasagiline; 136236-51-6; (R)-N-(2-Propynyl)-2,3-dihydroinden-1-amine | C12H13N |
| isocarboxazid; Isocarbonazid; Isocarboxazide . . . | C12H13N3O2 |
| METHSUXIMIDE; Mesuximide; Celontin . . . | C12H13NO2 |
| MEPHENYTOIN; Methoin; Phenantoin . . . | C12H14N2O2 |
| primidone; Primaclone; Mysoline . . . | C12H14N2O2 |
| sulfamethazine; Sulfadimidine; Sulfadimezine . . . | C12H14N4O2S |
| sulfacytine; Renoquid; 1-Ethyl-N-sulfanilylcytosine . . . | C12H14N4O3S |
| SULFADOXINE; Sulphadoxine; Sulforthomidine . . . | C12H14N4O4S |
| clofibrate; Atromid; Atromid-S . . . | C12H15ClO3 |
| albendazole; Valbazen; Albenza . . . | C12H15N3O2S |
| Entecavir; Baraclude; 142217-69-4 . . . | C12H15N5O3 |
| fenfluramine; Adifax; d-Fenfluramine . . . | C12H16F3N |
| Hexobarbital; Hexobarbitone; Evipal . . . | C12H16N2O3 |
| imipenem; Imipemide; Tienamycin . . . | C12H17N3O4S |
| thiamine; thiamin; Aneurin . . . | C12H17N4OS+ |
| CICLOPIROX; Loprox; Penlac . . . | C12H17NO2 |
| clenbuterol; Planipart; Clenbuterolum . . . | C12H18Cl2N2O |
| thiamylal; Thioseconal; Surital . . . | C12H18N2O2S |
| tolbutamide; Orinase; Diabetol . . . | C12H18N2O3S |
| midodrine; Midodrin; Midodrinum [INN-Latin] . . . | C12H18N2O4 |
| propofol; 2,6-DIISOPROPYLPHENOL; Diprivan . . . | C12H18O |
| procarbazine; Natulan; Ibenzmethyzin . . . | C12H19N3O |
| terbutaline; Bricanyl; Brican . . . | C12H19NO3 |
| PIRBUTEROL; pyrbuterol; Maxair . . . | C12H20N2O3 |
| sotalol; beta-Cardone; Sotalolum . . . | C12H20N2O3S |
| zanamivir; Relenza; Zanamavir . . . | C12H20N4O7 |
| memantine; Memantinum [INN-Latin]; Memantina [INN-Spanish] . . . | C12H21N |
| rimantadine; Remantadine; Riamantadine . . . | C12H21N |
| Brinzolamide; Azopt; Birnzolamide . . . | C12H21N3O5S3 |
| nizatidine; Axid; Acinon . . . | C12H21N5O2S2 |
| topiramate; Topamax; Epitomax . . . | C12H21NO8S |
| bithionol; Lorothidol; Bithin . . . | C12H6Cl4O2S |
| triclosan; 5-CHLORO-2-(2,4-DICHLOROPHENOXY)PHENOL; 3380-34-5 | C12H7Cl3O2 |
| leflunomide; Arava; 75706-12-6 . . . | C12H9F3N2O2 |
| milrinone; Primacor; 78415-72-2 . . . | C12H9N3O |
| NITAZOXANIDE; Alinia; 55981-09-4 . . . | C12H9N3O5S |
| thalidomide; Thalomid; Distaval . . . | C13H10N2O4 |
| Proflavin; Proflavine; 3,6-ACRIDINEDIAMINE . . . | C13H11N3 |
| tenoxicam; Mobiflex; Tilcotil . . . | C13H11N3O4S2 |
| ETHACRYNIC acid; Edecrin; Hydromedin . . . | C13H12Cl2O4 |
| fluconazole; Diflucan; Triflucan . . . | C13H12F2N6O |
| nimesulide; Mesulid; Nimed . . . | C13H12N2O5S |
| Monobenzone; 4-Benzyloxyphenol; 4-(Benzyloxy)phenol . . . | C13H12O2 |
| Lenalidomide; Revlimid; Revimid . . . | C13H13N3O3 |
| Cephacetrile; Cefacetrile; Cefacetril . . . | C13H13N3O6S |
| ceftizoxime; Ceftizoximum; Ceftizoxima . . . | C13H13N5O5S2 |
| Tacrine; Tetrahydroaminoacridine; Romotal . . . | C13H14N2 |
| Mephobarbital; Mebaral; Mephobarbitone . . . | C13H14N2O3 |
| Clorgiline; CLORGYLINE; Chlorgyline . . . | C13H15Cl2NO |
| glutethimide; Doriden; Noxiron . . . | C13H15NO2 |
| ketamine; Ketaject; Ketanest . . . | C13H16ClNO |
| Medetomidine; Medetomidinum [Latin]; Medetomidina [Spanish] . . . | C13H16N2 |
| aminoglutethimide; Cytadren; Orimeten . . . | C13H16N2O2 |
| Veliparib; ABT-888; 912444-00-9 . . . | C13H16N4O |
| moclobemide; Aurorix; Manerix . . . | C13H17ClN2O2 |
| selegiline; Emsam; Jumex . . . | C13H17N |
| Amfebutamone; bupropion; Amfebutamon . . . | C13H18ClNO |
| Heptabarbital; Heptabarb; Heptabarbitone . . . | C13H18N2O3 |
| pentoxifylline; Trental; Oxpentifylline . . . | C13H18N4O3 |
| ibuprofen; Motrin; Brufen . . . | C13H18O2 |
| CHLOROPROCAINE; Chloroprocain; Halestyn . . . | C13H19ClN2O2 |
| DIETHYLPROPION; Amfepramone; Diethylpropione . . . | C13H19NO |
| probenecid; Benemid; Probecid . . . | C13H19NO4S |
| prilocaine; Propitocaine; Citanest . . . | C13H20N2O |
| procaine; Novocaine; Duracaine . . . | C13H20N2O2 |
| valacyclovir; Valaciclovir; Valtrex . . . | C13H20N6O4 |
| PROCAINAMIDE; Novocainamide; Biocoryl . . . | C13H21N3O |
| Salbutamol; albuterol; Proventil . . . | C13H21NO3 |
| isoetharine; Isoetarin; Isoetarine . . . | C13H21NO3 |
| timolol; Betimol; Istalol . . . | C13H24N4O3S |
| hexachlorophene; Hexachlorofen; Hexachlorophen . . . | C13H6Cl6O2 |
| diflunisal; Dolobid; Dolobis . . . | C13H8F2O3 |
| niflumic acid; Nifluril; Landruma . . . | C13H9F3N2O2 |
| bromazepam; Compedium; Lectopam . . . | C14H10BrN3O |
| mitotane; Lysodren; Chlodithane . . . | C14H10Cl4 |
| Nitisinone; Orfadin; 104206-65-7 . . . | C14H10F3NO5 |
| dantrolene; Dantroleno; Dantrolenum [INN-Latin] . . . | C14H10N4O5 |
| Salsalate; sasapyrine; Disalcid . . . | C14H10O5 |
| diclofenac; Diclofenac acid; dichlofenac . . . | C14H11Cl2NO2 |
| meclofenamate; meclofenamic acid; Arquel . . . | C14H11Cl2NO2 |
| chlorthalidone; Chlortalidone; Phthalamudine . . . | C14H11ClN2O4S |
| Tolcapone; Tasmar; 134308-13-7 . . . | C14H11NO5 |
| LEVOSIMENDAN; Simdax; Levosimedan . . . | C14H12N6O |
| BENZYL BENZOATE; Ascabiol; Novoscabin . . . | C14H12O2 |
| oxybenzone; 2-HYDROXY-4-METHOXYBENZOPHENONE; 131-57-7 . . . | C14H12O3 |
| suprofen; Profenal; Maldocil . . . | C14H12O3S |
| tiaprofenic acid; Tiaprofensaeure; Surgam . . . | C14H12O3S |
| meloxicam; Mobic; Movalis . . . | C14H13N3O4S2 |
| cefdinir; Omnicef; CFDN . . . | C14H13N5O5S2 |
| ticlopidine; Ticlid; Ticlopidina . . . | C14H14ClNS |
| naphazoline; Naphthizine; Clearine . . . | C14H14N2 |
| metyrapone; Metopirone; Metopiron . . . | C14H14N2O |
| DL-Naproxen; Floginax; Naprius . . . | C14H14O3 |
| NAPROXEN; 22204-53-1; Naproxene . . . | C14H14O3 |
| ENTACAPONE; Comtan; Comtess . . . | C14H15N3O5 |
| Irosustat; 667-Coumate; 667 coumate . . . | C14H15NO5S |
| cyclothiazide; Anhydron; Aquirel . . . | C14H16ClN3O4S2 |
| IMIQUIMOD; Aldara; 99011-02-6 . . . | C14H16N4 |
| Opaviraline; GW420867X; Isopropyl (2s)-2-Ethyl-7-Fluoro-3-Oxo-3,4- | C14H17FN2O3 |
| Dihydroquinoxaline-1(2h)-Carboxylate . . . | |
| Frovatriptan; Frova; Miguard . . . | C14H17N3O |
| Fasudil; AT 877; Fasudil [INN] . . . | C14H17N3O2S |
| ibudilast; Ketas; Ke Tas . . . | C14H18N2O |
| METHOHEXITAL; Methohexitone; Brevital . . . | C14H18N2O3 |
| trimethoprim; Proloprim; Trimpex . . . | C14H18N4O3 |
| chlorambucil; Leukeran; Ambochlorin . . . | C14H19Cl2NO2 |
| famciclovir; Famvir; 104227-87-4 . . . | C14H19N5O4 |
| methylphenidate; Daytrana; Methylphenidan . . . | C14H19NO2 |
| pindolol; Visken; Prinodolol . . . | C14H20N2O2 |
| Vorinostat; SAHA; Zolinza . . . | C14H20N2O3 |
| sumatriptan; Imitrex; Sumatran . . . | C14H21N3O2S |
| oxamniquine; Vansil; Mansil . . . | C14H21N3O3 |
| tolazamide; Tolinase; Norglycin . . . | C14H21N3O3S |
| metoclopramide; Primperan; Reliveran . . . | C14H22ClN3O2 |
| lidocaine; Xylocaine; Lignocaine . . . | C14H22N2O |
| rivastigmine; ENA 713 free base; 123441-03-2 . . . | C14H22N2O2 |
| atenolol; Tenormin; Prenormine . . . | C14H22N2O3 |
| Valganciclovir; Cymeval; L-Valine, ester with ganciclovir . . . | C14H22N6O5 |
| Spectinomycin; actinospectacin; Trobicin . . . | C14H24N2O7 |
| ellagic acid; Benzoaric acid; Lagistase . . . | C14H6O8 |
| efavirenz; Sustiva; Stocrin . . . | C14H9ClF3NO2 |
| lorazepam; Ativan; Temesta . . . | C15H10Cl2N2O2 |
| clonazepam; Rivotril; Antelepsin . . . | C15H10ClN3O3 |
| phenindione; 2-Phenyl-1,3-indandione; Phenylindanedione . . . | C15H10O2 |
| oxazepam; Adumbran; Serax . . . | C15H11ClN2O2 |
| L-thyroxine; levothyroxine; synthroid . . . | C15H11I4NO4 |
| nitrazepam; Benzalin; Mogadon . . . | C15H11N3O3 |
| Bromfenac; Duract; Xibrom . . . | C15H12BrNO3 |
| carprofen; Rimadyl; Imadyl . . . | C15H12ClNO2 |
| liothyronine; triiodothyronine; Tresitope . . . | C15H12I3NO4 |
| carbamazepine; Tegretol; Carbamazepen . . . | C15H12N2O |
| phenytoin; 5,5-DIPHENYLHYDANTOIN; Diphenylhydantoin . . . | C15H12N2O2 |
| OXCARBAZEPINE; Trileptal; Oxcarbamazepine . . . | C15H12N2O2 |
| Lumiracoxib; Prexige; COX 189 . . . | C15H13ClFNO2 |
| flurbiprofen; Ansaid; Froben . . . | C15H13FO2 |
| piroxicam; Feldene; 36322-90-4 . . . | C15H13N3O4S |
| ketorolac; Ketoralac; Ketorolacum [Latin] . . . | C15H13NO3 |
| benzthiazide; Benzothiazide; Aquatag . . . | C15H14ClN3O4S3 |
| BENDROFLUMETHIAZIDE; Bendrofluazide; Naturetin . . . | C15H14F3N3O4S2 |
| flumazenil; Flumazepil; Romazicon . . . | C15H14FN3O3 |
| nevirapine; Viramune; 129618-40-2 . . . | C15H14N4O |
| ceftibuten; Cedax; Ceftibuteno . . . | C15H14N4O6S2 |
| fenoprofen; 2-(3-Phenoxyphenyl)propanoic acid; Nalfon . . . | C15H14O3 |
| mefenamic acid; Ponstel; Parkemed . . . | C15H15NO2 |
| tolmetin; Tolectin; Tolmetine . . . | C15H15NO3 |
| nabumetone; Relafen; Listran . . . | C15H16O2 |
| enoxacin; Penetrex; Comprecin . . . | C15H17FN4O3 |
| CEFPODOXIME; Cefpodoxima; Cefpodoximum . . . | C15H17N5O6S2 |
| cefmetazole; Cefmetazolum; Cefmetazolo . . . | C15H17N7O5S3 |
| mitomycin C; Mutamycin; Ametycine . . . | C15H18N4O5 |
| rizatriptan; Risatriptan; MK 462 free base . . . | C15H19N5 |
| acetohexamide; Dymelor; Gamadiabet . . . | C15H20N2O4S |
| fluvoxamine; Floxyfral; Dumirox . . . | C15H21F3N2O2 |
| Fencamfamin; Euvitol; Reactivan . . . | C15H21N |
| PRIMAQUINE; Neo-Quipenyl; Primachin . . . | C15H21N3O |
| physostigmine; Eserine; Antilirium . . . | C15H21N3O2 |
| gliclazide; Diamicron; Glimicron . . . | C15H21N3O3S |
| meperidine; Pethidine; Demerol . . . | C15H21NO2 |
| CAPECITABINE; Xeloda; 154361-50-9 . . . | C15H22FN3O6 |
| mepivacaine; Carbocaine; Scandicain . . . | C15H22N2O |
| Milnacipran; Midalcipran; Toledomin . . . | C15H22N2O |
| gemfibrozil; Lopid; Decrelip . . . | C15H22O3 |
| Mecillinam; Coactin; AMDINOCILLIN . . . | C15H23N3O3S |
| ciclacillin; CYCLACILLIN; Bastcillin . . . | C15H23N3O4S |
| sulpiride; Aiglonyl; Sulpirid . . . | C15H23N3O4S |
| alprenolol; Alfeprol; Yobir . . . | C15H23NO2 |
| oxprenolol; Coretai; dl-Oxprenolol . . . | C15H23NO3 |
| Lisdexamfetamine; NRP104; 608137-32-2 . . . | C15H25N3O |
| metoprolol; Lopressor; Seloken . . . | C15H25NO3 |
| Marimastat; Marimastat [USAN]; BB-2516 . . . | C15H29N3O5 |
| fludiazepam; Erispan; Fludiazepamum [INN-Latin] . . . | C16H12ClFN2O |
| Benoxaprofen; Oraflex; Inflamid . . . | C16H12ClNO3 |
| flunitrazepam; Rohypnol; Narcozep . . . | C16H12FN3O3 |
| anisindione; Miradon; Anisin indandione . . . | C16H12O3 |
| tioconazole; Vagistat-1; Trosyl . . . | C16H13Cl3N2OS |
| diazepam; Valium; Ansiolisina . . . | C16H13ClN2O |
| mazindol; Mazanor; Sanorex . . . | C16H13ClN2O |
| Clobazam; Chlorepin; Urbanyl . . . | C16H13ClN2O2 |
| temazepam; Crisonar; Restoril . . . | C16H13ClN2O2 |
| mebendazole; Vermox; Telmin . . . | C16H13N3O3 |
| chlordiazepoxide; Chlozepid; Elenium . . . | C16H14ClN3O |
| lansoprazole; Bamalite; Monolitum . . . | C16H14F3N3O2S |
| Voriconazole; Vfend; 137234-62-9 . . . | C16H14F3N5O |
| TOLRESTAT; Alredase; Tolrestatum . . . | C16H14F3NO3S |
| amlexanox; Aphthasol; Amoxanox . . . | C16H14N2O4 |
| ketoprofen; Orudis; Profenid . . . | C16H14O3 |
| clotiazepam; Trecalmo; Rizen . . . | C16H15ClN2OS |
| pantoprazole; Protonix; Pantozol . . . | C16H15F2N3O4S |
| Sitagliptin; Xelevia; Januvia . . . | C16H15F6N5O |
| Fosphenytoin; Prodilantin; Fosfenitoina [INN-Spanish] . . . | C16H15N2O6P |
| epinastine; Epinastinum [Latin]; Epinastina [Spanish] . . . | C16H15N3 |
| Tanaproget; 1zuc; NSP-989 . . . | C16H15N3OS |
| indapamide; Tertensif; Arifon . . . | C16H16ClN3O3S |
| metolazone; Zaroxolyn; Mykrox . . . | C16H16ClN3O3S |
| clopidogrel; Plavix; (S)-Clopidogrel . . . | C16H16ClNO2S |
| fenoldopam; Corlopam; Fenoldopamum [Latin] . . . | C16H16ClNO3 |
| Febuxostat; 144060-53-7; Uloric . . . | C16H16N2O3S |
| cephalothin; Cefalotin; Cephalothinum . . . | C16H16N2O6S2 |
| Zimeldine; Zimelidine; cis-Zimelidine . . . | C16H17BrN2 |
| cephalexin; Cefalexin; Cephacillin . . . | C16H17N3O4S |
| cefoxitin; Mefoxitin; Mefoxin . . . | C16H17N3O7S2 |
| norfloxacin; Noroxin; Chibroxin . . . | C16H18FN3O3 |
| Nomifensine; nomifensin; Linamiphen . . . | C16H18N2 |
| penicillin g; Benzylpenicillin; Pencillin G . . . | C16H18N2O4S |
| penicillin v; Phenomycilline; Beromycin . . . | C16H18N2O5S |
| BROMPHENIRAMINE; Dimetane; Bromfeniramina . . . | C16H19BrN2 |
| chlorpheniramine; Chlorphenamine; Chlor-trimeton . . . | C16H19ClN2 |
| carbinoxamine; Paracarbinoxamine; Allergefon . . . | C16H19ClN2O |
| cephradine; Cefradine; Anspor . . . | C16H19N3O4S |
| ampicillin; Omnipen; Ampicillin acid . . . | C16H19N3O4S |
| amoxicillin; Amoxycillin; Amoxicilline . . . | C16H19N3O5S |
| Tivirapine; CHEMBL58711; NSC636661 . . . | C16H20ClN3S |
| linezolid; Zyvox; Zyvoxid . . . | C16H20FN3O4 |
| pheniramine; Prophenpyridamine; Tripoton . . . | C16H20N2 |
| TORSEMIDE; Torasemide; Demadex . . . | C16H20N4O3S |
| Tasocitinib; Tofacitinib; 477600-75-2 . . . | C16H20N6O |
| tripelennamine; Pyribenzamine; Tripelennamin . . . | C16H21N3 |
| Ramelteon; Rozerem; TAK-375 . . . | C16H21NO2 |
| propranolol; Propanolol; beta-Propranolol . . . | C16H21NO2 |
| Remoxipride; Romoxipride; Remoxipridum [INN-Latin] . . . | C16H23BrN2O3 |
| HEXYLCAINE; Hexylcainum [INN-Latin]; Hexilcaina [INN-Spanish] . . . | C16H23NO2 |
| oxymetazoline; Oxymethazoline; Oxylazine . . . | C16H24N2O |
| ropinirole; ReQuip; ropinirol . . . | C16H24N2O |
| MOLINDONE; Moban; (+−)-Molindone . . . | C16H24N2O2 |
| CARTEOLOL; Carteololum; Carteololum [INN-Latin] . . . | C16H24N2O3 |
| Desvenlafaxine; O-Desmethylvenlafaxine; 93413-62-8 . . . | C16H25NO2 |
| Tramadol; Ultram; Ralivia flashtab . . . | C16H25NO2 |
| ESMOLOL; (+−)-Esmolol; Esmolol [INN:BAN] . . . | C16H25NO4 |
| proparacaine; Ophthaine; Proxymetacaine . . . | C16H26N2O3 |
| Cilastatin; Cilastatina; Cilastatine . . . | C16H26N2O5S |
| oseltamivir; Tamiflu; Tamvir . . . | C16H28N2O4 |
| palmitic acid; Hexadecanoic acid; Cetylic acid . . . | C16H32O2 |
| Zenarestat; Zenarestatum; Zenarestatum [INN-Latin] . . . | C17H11BrClFN2O4 |
| quazepam; Doral; Dormalin . . . | C17H11ClF4N2S |
| letrozole; Femara; Letrozol . . . | C17H11N5 |
| triazolam; Halcion; Clorazolam . . . | C17H12Cl2N4 |
| Halazepam; Paxipam; Pacinone . . . | C17H12ClF3N2O |
| alprazolam; Xanax; Cassadan . . . | C17H13ClN4 |
| celecoxib; Celebrex; Celebra . . . | C17H14F3N3O2S |
| ROSOXACIN; Acrosoxacin; Eradacil . . . | C17H14N2O3 |
| rofecoxib; Vioxx; Ceoxx . . . | C17H14O4S |
| zaleplon; Sonata; 151319-34-5 . . . | C17H15N5O |
| amoxapine; Asendin; Demolox . . . | C17H16ClN3O |
| Lariam; mefloquine; Mefloquinone . . . | C17H16F6N2O |
| sertraline; Lustral; Zoloft . . . | C17H17Cl2N |
| zopiclone; Imovane; Amoban . . . | C17H17ClN6O3 |
| cephapirin; Cefapirin; Cephapirine . . . | C17H17N3O6S2 |
| fluoxetine; Pulvules; Portal . . . | C17H18F3NO |
| ciprofloxacin; Ciprobay; Cipro . . . | C17H18FN3O3 |
| piretanide; Tauliz; Arelix (TN) . . . | C17H18N2O5S |
| nifedipine; Procardia; Adalat . . . | C17H18N2O6 |
| carbenicillin; Pyopen; Carboxybenzylpenicillin . . . | C17H18N2O6S |
| alosetron; Lotronex; Alosetron HCl . . . | C17H18N4O |
| chlorpromazine; Largactil; Thorazine . . . | C17H19ClN2S |
| lomefloxacin; Lomefloxacine; Lomefloxacino . . . | C17H19F2N3O3 |
| antazoline; Antasten; Phenazoline . . . | C17H19N3 |
| mirtazapine; Remeron; Remergil . . . | C17H19N3 |
| phentolamine; Regitine; Rogitine . . . | C17H19N3O |
| omeprazole; Prilosec; Antra . . . | C17H19N3O3S |
| anastrozole; Arimidex; anastrazole . . . | C17H19N5 |
| Piritrexim; Piritrexime [French]; Piritreximum [Latin] . . . | C17H19N5O2 |
| hydromorphone; Dihydromorphinone; Dimorphone . . . | C17H19NO3 |
| oxymorphone; Numorphan; Oximorphonum . . . | C17H19NO4 |
| Bromdiphenhydramine; bromodiphenhydramine; Bromazine . . . | C17H20BrNO |
| CHLOPHEDIANOL; Chlofedanol; Dencyl . . . | C17H20ClNO |
| flecainide; Flecaine; Tambocor . . . | C17H20F6N2O3 |
| Pefloxacin; Pefloxacine; Pefloxacinum . . . | C17H20FN3O3 |
| tropicamide; Tropicacyl; Mydriacyl . . . | C17H20N2O2 |
| bumetanide; Bumex; Fordiuran . . . | C17H20N2O5S |
| methicillin; Meticillin; Methicillinum . . . | C17H20N2O6S |
| promazine; Protactyl; Sinophenin . . . | C17H20N2S |
| promethazine; Proazamine; Diphergan . . . | C17H20N2S |
| olanzapine; Zyprexa; Zyprexa Zydis . . . | C17H20N4S |
| mycophenolic acid; Melbex; Mycophenolate . . . | C17H20O6 |
| diphenhydramine; Benadryl; Alledryl . . . | C17H21NO |
| Atomoxetine; Tomoxetine; Tomoxetinum . . . | C17H21NO |
| etodolac; Etodolic acid; Lodine . . . | C17H21NO3 |
| ritodrine; Yutopar; Ritodrina [INN-Spanish] . . . | C17H21NO3 |
| fenoterol; Phenoterol; Berotec . . . | C17H21NO4 |
| doxylamine; Dossilamina [DCIT]; Doxylaminum [INN-Latin] . . . | C17H22N2O |
| Emivirine; Coactinon; MKC-442 . . . | C17H22N2O3 |
| minaprine; Cantor; Brantur . . . | C17H22N4O |
| PF-734200; PF-00734200; 869490-23-3 . . . | C17H24F2N6O |
| PHENCYCLIDINE; Angel dust; 1-(1-Phenylcyclohexyl)piperidine . . . | C17H25N |
| Almotriptan; Axert; Almogran . . . | C17H25N3O2S |
| naratriptan; Naramig; Amerge . . . | C17H25N3O2S |
| cyclopentolate; Cyclogyl; Ciclopentolato . . . | C17H25NO3 |
| LEVOBUNOLOL; Betagan; Akbeta . . . | C17H25NO3 |
| sibutramine; Reductil; Medaria . . . | C17H26ClN |
| emedastine; Emadine; Emedastinum [INN-Latin] . . . | C17H26N4O |
| amisulpride; Solian; Aminosultopride . . . | C17H27N3O4S |
| venlafaxine; Elafax; Venlafaxina . . . | C17H27NO2 |
| Metipranolol; Trimepranol; Disorat . . . | C17H27NO4 |
| nadolol; Corgard; Solgol . . . | C17H27NO4 |
| Oxybuprocaine; Benoxinate; Oxybucaine . . . | C17H28N2O3 |
| Lidorestat; IDD-676; IDD-000676-01 . . . | C18H11F3N2O2S |
| midazolam; Dormicum; Versed . . . | C18H13ClFN3 |
| Cinolazepam; Gerodorm; Cinolazepamum . . . | C18H13ClFN3O2 |
| miconazole; Monistat; Daktarin IV . . . | C18H14Cl4N2O |
| (R)-Bicalutamide; R-BICALUTAMIDE; CHEBL3090 . . . | C18H14F4N2O4S |
| econazole; Ecostatin; Pevaryl . . . | C18H15Cl3N2O |
| Etoricoxib; Arcoxia; Tauxib . . . | C18H15ClN2O2S |
| Sitaxsentan; Sitaxentan; Thelin . . . | C18H15ClN2O6S2 |
| oxaprozin; Daypro; Deflam . . . | C18H15NO3 |
| PHENPROCOUMON; Phenprocoumarol; Liquamar . . . | C18H16O3 |
| tranilast; Rizaben; Tranilastum . . . | C18H17NO5 |
| loxapine; Cloxazepine; Dibenzoazepine . . . | C18H18ClN3O |
| cefamandole; Cefadole; Cefamandolum . . . | C18H18N6O5S2 |
| ceftriaxone; Biotrakson; Cefatriaxone . . . | C18H18N8O7S3 |
| dienestrol; Dehydrostilbestrol; Dienoestrol . . . | C18H18O2 |
| felodipine; Plendil; Perfudal . . . | C18H19Cl2NO4 |
| clozapine; Leponex; Fazaclo . . . | C18H19ClN4 |
| triflupromazine; Fluopromazine; Siquil . . . | C18H19F3N2S |
| ondansetron; Zofran; Zophren . . . | C18H19N3O |
| rosiglitazone; Avandia; Rosiglizole . . . | C18H19N3O3S |
| cefprozil; Cefprozil anhydrous; Brisoral . . . | C18H19N3O5S |
| Cefaloglycin; Cephaloglycine; CEPHALOGLYCIN . . . | C18H19N3O6S |
| duloxetine; Cymbalta; Yentreve . . . | C18H19NOS |
| LEVOFLOXACIN; Levaquin; Quixin . . . | C18H20FN3O4 |
| mianserin; Mianserine; Mianserina . . . | C18H20N2 |
| nitrendipine; Baypress; Bayotensin . . . | C18H20N2O6 |
| METHDILAZINE; Tacaryl; Methodilazine . . . | C18H20N2S |
| diethylstilbestrol; Stilbestrol; Distilbene . . . | C18H20O2 |
| rabeprazole; Rebeprazole sodium; 117976-89-3 . . . | C18H21N3O3S |
| codeine; Methylmorphine; Codicept . . . | C18H21NO3 |
| HYDROCODONE; Dihydrocodeinone; Hydrocodon . . . | C18H21NO3 |
| oxycodone; Dihydrone; Dihydroxycodeinone . . . | C18H21NO4 |
| phenoxybenzamine; Dibenyline; Dibenzyran . . . | C18H22ClNO |
| desipramine; Norimipramine; Desmethylimipramine . . . | C18H22N2 |
| CYCLIZINE; Marezine; Nautazine . . . | C18H22N2 |
| trimeprazine; Methylpromazine; Alimemazine . . . | C18H22N2S |
| estrone; folliculin; Theelin . . . | C18H22O2 |
| cefotiam; Cefotiamum; Cefotiamum [INN-Latin] . . . | C18H23N9O4S3 |
| orphenadrine; Disipal; o-Methyldiphenhydramine . . . | C18H23NO |
| dobutamine; racemic-Dobutamine; Dobutrex . . . | C18H23NO3 |
| Genesa; ARBUTAMINE; Arbutaminum . . . | C18H23NO4 |
| Kytril; Granisetron base; Granisetronum [INN-Latin] . . . | C18H24N4O |
| estradiol; beta-Estradiol; 17beta-Estradiol . . . | C18H24O2 |
| estriol; Oestriol; Estratriol . . . | C18H24O3 |
| Saxagliptin; Onglyza; BMS-477118 . . . | C18H25N3O2 |
| d-Methorphan; dextromethorphan; Delsym . . . | C18H25NO |
| chloroquine; Aralen; Artrichin . . . | C18H26ClN3 |
| hydroxychloroquine; Oxichlorochinum; Oxychlorochin . . . | C18H26ClN3O |
| dyclonine; Dyclocaine; Dyclocainum . . . | C18H27NO2 |
| bupivacaine; Marcaine; Sensorcaine . . . | C18H28N2O |
| Levobupivacaine; L(−)-Bupivacaine; (S)-bupivacaine . . . | C18H28N2O |
| acebutolol; Acetobutolol; Neptal . . . | C18H28N2O4 |
| Bambuterol; Bambec; Bambuterolum . . . | C18H29N3O5 |
| penbutolol; Betapressin; Levatol . . . | C18H29NO2 |
| betaxolol; Betoptic; Betaxololum . . . | C18H29NO3 |
| BISOPROLOL; Concor; Zebeta . . . | C18H31NO4 |
| KANAMYCIN A; kanamycin; Kanamycin Base . . . | C18H36N4O11 |
| ZOPOLRESTAT; CP-73850; 110703-94-1 . . . | C19H12F3N3O3S |
| dicumarol; dicoumarol; Bishydroxycoumarin . . . | C19H12O6 |
| ACENOCOUMAROL; Acenocoumarin; Nicoumalone . . . | C19H15NO6 |
| indomethacin; indometacin; Indocin . . . | C19H16ClNO4 |
| warfarin; Coumafene; Coumadin . . . | C19H16O4 |
| dicloxacillin; Dicloxacycline; Dynapen . . . | C19H17Cl2N3O5S |
| Butoconazole; Butoconazolum; Butoconazol . . . | C19H17Cl3N2S |
| FluCloxacillin; FLOXACILLIN; Floxapen . . . | C19H17ClFN3O5S |
| prazepam; Demetrin; Lysanxia . . . | C19H17ClN2O |
| tolnaftate; Tinactin; Aftate . . . | C19H17NOS |
| Cloxacillin; Cloxacilline; Cloxacillinum . . . | C19H18ClN3O5S |
| Rivaroxaban; Xarelto; 366789-02-8 . . . | C19H18ClN3O5S |
| ADINAZOLAM; Deracyn; Adinazolamum . . . | C19H18ClN5 |
| Desloratadine; Clarinex; Descarboethoxyloratadine . . . | C19H19ClN2 |
| Phenindamine; Thephorin; Nolahist . . . | C19H19N |
| oxacillin; Oxazocillin; MPI-penicillin . . . | C19H19N3O5S |
| nilvadipine; Escor; Nivadil . . . | C19H19N3O6 |
| folic acid; Folate; Folvite . . . | C19H19N7O6 |
| ketotifen; Ketotifene; Ketotifene [INN-French] . . . | C19H19NOS |
| bezafibrate; Bezalip; Cedur . . . | C19H20ClNO4 |
| paroxetine; Paxil; Aropax . . . | C19H20FNO3 |
| phenylbutazone; Butazolidin; Diphenylbutazone . . . | C19H20N2O2 |
| Pioglitazone; Actos; Pioglitazona . . . | C19H20N2O3S |
| nortriptyline; Sensaval; Noritren . . . | C19H21N |
| protriptyline; Amimetilina; Protryptyline . . . | C19H21N |
| zolpidem; Lorex; Zolpidemum [Latin] . . . | C19H21N3O |
| isradipine; Dynacirc; Lomir . . . | C19H21N3O5 |
| pirenzepine; Pirenzepin; Gastrozepin . . . | C19H21N5O2 |
| prazosin; Furazosin; Minipress . . . | C19H21N5O4 |
| naloxone; I-Naloxone; n-Allylnoroxymorphone . . . | C19H21NO4 |
| trazodone; Beneficat; Trazalon . . . | C19H22ClN5O |
| sparfloxacin; Zagam; Cl-978 . . . | C19H22F2N4O3 |
| Grepafloxacin; Raxar; Grepafloxacin [INN] . . . | C19H22FN3O3 |
| Gatifloxacin; Tequin; Zymar . . . | C19H22FN3O4 |
| Actidil; triprolidine; Myidyl . . . | C19H22N2 |
| Acepromethazine; Aceprometazine; Aceprometazina . . . | C19H22N2OS |
| clomipramine; Chlorimipramine; Hydiphen . . . | C19H23ClN2 |
| Ergometrine; ERGONOVINE; Ergobasine . . . | C19H23N3O2 |
| hetacillin; Versapen; Hetacillinum . . . | C19H23N3O4S |
| trimetrexate; Cl-898; Trimetrexatum [INN-Latin] . . . | C19H23N5O3 |
| diphenylpyraline; Allergen; Belfene . . . | C19H23NO |
| Reboxetine; Esreboxetine; Davedax . . . | C19H23NO3 |
| imipramine; Imidobenzyle; Melipramine . . . | C19H24N2 |
| praziquantel; Biltricide; Droncit . . . | C19H24N2O2 |
| labetalol; Labetolol; Albetol . . . | C19H24N2O3 |
| formoterol; Oxis; Foradile . . . | C19H24N2O4 |
| ethopropazine; Parsidol; Profenamine . . . | C19H24N2S |
| pentamidine; Pentacarinat; 100-33-4 . . . | C19H24N4O2 |
| cefepime; Maxipime; Axepim . . . | C19H24N6O5S2 |
| testolactone; Teslac; Fludestrin . . . | C19H24O3 |
| Bortezomib; PS-341; 179324-69-7 . . . | C19H25BN4O4 |
| terazosin; Terazosine; Flumarc . . . | C19H25N5O4 |
| levallorphan; Naloxiphan; Lorfan . . . | C19H25NO |
| pergolide; Permax; Pergolidum [INN-Latin] . . . | C19H26N2S |
| Dofetilide; Tikosyn; Dofetilida . . . | C19H27N3O5S2 |
| Dapiprazole; Dapiprazol; Glamidolo . . . | C19H27N5 |
| alfuzosin; Alfuzosinum; Alfusosine . . . | C19H27N5O4 |
| pentazocine; Pentazocaine; Liticon . . . | C19H27NO |
| TETRABENAZINE; Nitoman; Rubigen . . . | C19H27NO3 |
| testosterone; Androderm; Testoderm . . . | C19H28O2 |
| cycrimine; Pagitane; Cicrimina . . . | C19H29NO |
| procyclidine; Tricyclamol; Arpicolin . . . | C19H29NO |
| dipivefrin; Dipivefrine; Propine . . . | C19H29NO5 |
| OXANDROLONE; Oxandrin; Anavar . . . | C19H30O3 |
| PERINDOPRIL; Coversyl; ACEON . . . | C19H32N2O5 |
| PERHEXILINE; Perhexilene; 2-(2,2-Dicyclohexylethyl)piperidine . . . | C19H35N |
| dicyclomine; Dicycloverin; Dicycloverine . . . | C19H35NO2 |
| IODIPAMIDE; Adipiodone; Cholografin . . . | C20H14I6N2O6 |
| Etravirine; Intelence; TMC-125 . . . | C20H15BrN6O |
| Sertaconazole; Sertaconazol [Spanish]; Sertaconazolum [Latin] . . . | C20H15Cl3N2OS |
| Trovafloxacin; Trovan; TVFX . . . | C20H15F3N4O3 |
| moxalactam; Latamoxef; Lamoxactam . . . | C20H20N609S |
| fenofibrate; Lipanthyl; Antara . . . | C20H21ClO4 |
| Escitalopram; (S)-Citalopram; S(+)-Citalopram . . . | C20H21FN2O |
| Citalopram; Nitalapram; Cipram . . . | C20H21FN2O |
| cyclobenzaprine; Flexeril; Proheptatriene . . . | C20H21N |
| Alimta; Pemetrexed [INN:BAN]; HSDB 7316 . . . | C20H21N5O6 |
| CEFORANIDE; Precef; Ceforanido . . . | C20H21N7O6S2 |
| amodiaquine; Amodiaquin; Camoquine . . . | C20H22ClN3O |
| AZATADINE; Optimine; Azatadine maleate . . . | C20H22N2 |
| methotrexate; Amethopterin; Rheumatrex . . . | C20H22N8O5 |
| amitriptyline; Damilen; Elavil . . . | C20H23N |
| maprotiline; Deprilept; Maprotylina [Polish] . . . | C20H23N |
| naltrexone; Celupan; Vivitrex . . . | C20H23NO4 |
| Methixart; Metixene; METHIXENE . . . | C20H23NS |
| prochlorperazine; Compazine; Prochlorperazin . . . | C20H24ClN3S |
| DIMETHINDENE; Dimetindene; Forhistal . . . | C20H24N2 |
| Ricainid; IN DECAIN IDE; Ricainide . . . | C20H24N2O |
| nisoldipine; Sular; Nisocor . . . | C20H24N2O6 |
| LUCANTHONE; Lucanthon; Tixantone . . . | C20H24N2OS |
| propiomazine; Phenoctyl; Propionylpromethazine . . . | C20H24N2OS |
| ethinyl estradiol; Ethynylestradiol; Ethinyloestradiol . . . | C20H24O2 |
| EXEMESTANE; Aromasin; Exemestance . . . | C20H24O2 |
| Sobetirome; GC-1; Q.RX-431 . . . | C20H24O4 |
| amlodipine; Amlodis; Amlocard . . . | C20H25ClN2O5 |
| Lysergide; Lysergamid; LYSERGIC acid DIETHYLAMIDE . . . | C20H25N3O |
| methylergonovine; Methylergobasin; Methylergometrin . . . | C20H25N3O2 |
| tiagabine; Tiagabinum [INN-Latin]; Tiagabina [INN-Spanish] . . . | C20H25NO2S2 |
| Cilomilast; Ariflo; 153259-65-5 . . . | C20H25NO4 |
| trimipramine; Trimeprimine; Sapilent . . . | C20H26N2 |
| LISURIDE; Lysuride; lisuride maleate . . . | C20H26N4O |
| norethindrone; Norethisterone; Micronor . . . | C20H26O2 |
| cilostazol; Pletal; Pletaal . . . | C20H27N5O2 |
| Glisoxepide; Glisoxepid; Glisepin . . . | C20H27N5O5S |
| Trilostane; Modrastane; Modrenal . . . | C20H27NO3 |
| Bevantolol; Bevantololum; Bevantololum [INN-Latin] . . . | C20H27NO4 |
| oxyphencyclimine; Manir; Oxiphencycliminum . . . | C20H28N2O3 |
| REMIFENTANIL; Remifentanyl; Ultiva . . . | C20H28N2O5 |
| TAMSULOSIN; Flomax; 106133-20-4 . . . | C20H28N2O5S |
| Ilomastat; Galardin; GM 6001 . . . | C20H28N4O4 |
| Retinoic acid; tretinoin; Vitamin A acid . . . | C20H28O2 |
| fluoxymesterone; Fluoxymestrone; Halotestin . . . | C20H29FO3 |
| dibucaine; Cinchocaine; Nupercainal . . . | C20H29N3O2 |
| retinol; Vitamin A; all-trans-Retinol . . . | C20H300 |
| Timnodonic acid; Eicosapentaenoic acid; Icosapent . . . | C20H3002 |
| trihexyphenidyl; Benzhexol; Triphenidyl . . . | C20H31NO |
| ADEFOVIR DIPIVOXIL; Hepsera; Preveon . . . | C20H32N5O8P |
| Dinoprostone; Prostaglandin E2; PGE2 . . . | C20H32O5 |
| dinoprost; prostaglandin F2alpha; amoglandin . . . | C20H34O5 |
| alprostadil; Prostaglandin El; Caverject . . . | C20H34O5 |
| Ibutilide; Ibutilidum [INN-Latin]; Corvert . . . | C20H36N2O3S |
| Sorafenib; Nexavar; 284461-73-0 . . . | C21H16ClF3N4O3 |
| Permethrin; Transpermethrin; Elimite . . . | C21H20Cl2O3 |
| Flavopiridol; Alvocidib; FLAVO . . . | C21H20ClNO5 |
| Ziprasidone; Geodon; Zeldox . . . | C21H21ClN4OS |
| CYPROHEPTADINE; Eiproheptadine; Periactin . . . | C21H21N |
| Naftifine; Naftifin; Naftifina . . . | C21H21N |
| tazarotene; Tazorac; Zorac . . . | C21H21NO2S |
| nafcillin; Naphcillin; Nafcilin-1 . . . | C21H22N2O5S |
| Raltitrexed; Tomudex; ZD1694 . . . | C21H22N4O6S |
| flurazepam; Dalmane; Dalmadorm . . . | C21H23ClFN3O |
| haloperidol; Haldol; Aloperidol . . . | C21H23ClFNO2 |
| periClazine; PERICYAZINE; ProperiClazine . . . | C21H23N3OS |
| Olopatadine; Patanol; Opatanol . . . | C21H23NO3 |
| trifluoperazine; Trifluperazine; Trifluoroperazine . . . | C21H24F3N3S |
| moxifloxaCln; Vigamox; Avelox I.V. . . . | C21H24FN3O4 |
| cetirizine; Virlix; Cetryn . . . | C21H25ClN2O3 |
| terbinafine; Lamisil; Lamasil . . . | C21H25N |
| quetiapine; Seroquel; 111974-69-7 . . . | C21H25N3O2S |
| MEZLOCILLIN; Mezlin; Mezlocilina . . . | C21H25N5O8S2 |
| perphenazine; Trilafon; Perfenazine . . . | C21H26ClN3OS |
| clemastine; Mecloprodin; Meclastin . . . | C21H26ClNO |
| nimodipine; Nimotop; Periplum . . . | C21H26N2O7 |
| mesoridazine; Lidanar; Serentil . . . | C21H26N2OS2 |
| thioridazine; Mellaril; Melleril . . . | C21H26N2S2 |
| mestranol; Devocin; Norquen . . . | C21H26O2 |
| prednisone; Deltasone; Meticorten . . . | C21H26O5 |
| hydroxyzine; Hychotine; Hydroxine . . . | C21H27ClN2O2 |
| triamcinolone; Fluoxyprednisolone; Aristocort . . . | C21H27FO6 |
| bacampicillin; Penglobe; Bacampicilline . . . | C21H27N3O7S |
| glipizide; Glucotrol; Glydiazinamide . . . | C21H27N5O4S |
| Difenidol; DIPHENIDOL; Vontrol . . . | C21H27NO |
| methadone; Adanon; Dolophine . . . | C21H27NO |
| propafenone; Rythmol; Propafenona . . . | C21H27NO3 |
| Levonorgestrel; Mirena; D-Norgestrel . . . | C21H28O2 |
| dydrogesterone; Isopregnenone; Hydrogesterone . . . | C21H28O2 |
| prednisolone; Meticortelone; Metacortandralone . . . | C21H28O5 |
| ALDOSTERONE; Aldocortin; Electrocortin . . . | C21H28O5 |
| fludrocortisone; Fluohydrisone; Fluorocortisol . . . | C21H29FO5 |
| disopyramide; Dicorantil; Ritmodan . . . | C21H29N3O |
| biperiden; Akineton; Biperidene . . . | C21H29NO |
| Dronabinol; Marinol; Deltanyne . . . | C21H30O2 |
| progesterone; Crinone; Luteohormone . . . | C21H30O2 |
| hydrocortisone; Cortisol; Cortef . . . | C21H30O5 |
| lisinopril; Prinivil; Zestril . . . | C21H31N3O5 |
| buspirone; Ansial; Buspirona . . . | C21H31N5O2 |
| ALFENTANIL; Alfentanyl; Alfenta . . . | C21H32N6O3 |
| oxyphenonium; Methacin; Oxiphenonum . . . | C21H34NO3+ |
| Hemabate; C21H36O5; 15-Methyl-PGF2alpha . . . | C21H36O5 |
| clotrimazole; Lotrimin; Mycelex . . . | C22H17ClN2 |
| bifonazole; Mycospor; Trifonazole . . . | C22H18N2 |
| Rilpivirine; TMC278; TMC 278 . . . | C22H18N6 |
| Tadalafil; Cialis; Tadanafil . . . | C22H19N3O4 |
| Rimonabant; Acomplia; Zimulti . . . | C22H21Cl3N4O |
| Cinacalcet; 226256-56-0; Cinacalcet [INN] . . . | C22H22F3N |
| droperidol; Droleptan; Inapsine . . . | C22H22FN3O2 |
| ceftazidime; Fortaz; ceftazidima . . . | C22H22N6O7S2 |
| podophyllotoxin; Podofilox; Condylox . . . | C22H22O8 |
| loratadine; Claritin; Loratidine . . . | C22H23ClN2O2 |
| losartan; Lortaan; Hyzaar . . . | C22H23ClN6O |
| Nesbuvir; HCV-796; Nesbuvir (USAN) . . . | C22H23FN2O5S |
| Erlotinib; 183321-74-6; N-(3-Ethynylphenyl)-6,7-bis(2- | C22H23N3O4 |
| methoxyethoxy)quinazolin-4-amine . . . | |
| Vandetanib; Zactima; 443913-73-3 . . . | C22H24BrFN4O2 |
| Gefitinib; Iressa; 184475-35-2 . . . | C22H24ClFN4O3 |
| azelastine; Azelastinum [INN-Latin]; Azelastina [INN-Spanish] . . . | C22H24ClN3O |
| domperidone; Motilium; Nauzelin . . . | C22H24ClN5O2 |
| Zuclopenthixol; Clopixol; Zuclopentixol [Spanish] . . . | C22H25ClN2OS |
| Nebivolol; Nebilet; Lobivon . . . | C22H25F2NO4 |
| MORICIZINE; Moracizine; Ethmozine . . . | C22H25N3O4S |
| Ertapenem; Invanz; Ertapenem (INN) . . . | C22H25N3O7S |
| Tamibarotene; Am 80; Tamibaro . . . | C22H25NO3 |
| Dasatinib; Sprycel; BMS-354825 . . . | C22H26ClN7O2S |
| FLUPHENAZINE; Triflumethazine; Fluorfenazine . . . | C22H26F3N3OS |
| Eletriptan; Relpax; eletriptanum . . . | C22H26N2O2S |
| diltiazem; Cardizem; Anoheal . . . | C22H26N2O4S |
| cyproterone; Gen-Cyproterone; CyPat . . . | C22H27ClO3 |
| Sunitinib; Sutent; SU-11248 . . . | C22H27FN4O2 |
| danazol; Danocrine; Cyclomen . . . | C22H27NO2 |
| Amineptine; Amineptin; Amineptino . . . | C22H27NO2 |
| fentanyl; Duragesic; Fentanest . . . | C22H28N2O |
| ANILERIDINE; Alidine; Leritin . . . | C22H28N2O2 |
| Etonitazene; Etonitazine; Etonitazinum . . . | C22H28N4O3 |
| mitoxantrone; Mitozantrone; Dihydroxyanthraquinone . . . | C22H28N4O6 |
| Delavirdine; Rescriptor; 136817-59-9 . . . | C22H28N6O3S |
| desoximetasone; Topicort; Desoximetasona . . . | C22H29FO4 |
| fluoromethoIone; Oxylone; FluormethoIone . . . | C22H29FO4 |
| dexamethasone; Maxidex; Dexamethazone . . . | C22H29FO5 |
| paramethasone; Cassenne; Cortiden . . . | C22H29FO5 |
| betamethasone; Flubenisolone; Betadexamethasone . . . | C22H29FO5 |
| thiethylperazine; Torecan; ETHYLTHIOPERAZINE . . . | C22H29N3S2 |
| chlorhexidine; Tubulicid; Hexadol . . . | C22H30Cl2N10 |
| APRINDINE; Aprinidine; Aprindin . . . | C22H30N2 |
| SUFENTANIL; Chronogesic; Sufentanyl . . . | C22H30N2O2S |
| Spirapril; Renormax; Sandopril . . . | C22H30N2O5S2 |
| sildenafil; Revatio; 139755-83-2 . . . | C22H30N6O4S |
| DESOGESTREL; Cerazette; desogen . . . | C22H30O |
| methylprednisolone; Medrol; Medrone . . . | C22H30O5 |
| cilazapril; Inhibace; Cilazaprilum [Latin] . . . | C22H31N3O5 |
| oxybutynin; Oxytrol; Ditropan . . . | C22H31NO3 |
| benzquinamide; Quantril; Emete-con . . . | C22H32N2O5 |
| medrysone; Hydroxymesterone; Medritonic . . . | C22H32O3 |
| testosterone propionate; Agovirin; Testex . . . | C22H32O3 |
| forskolin; Colforsin; Coleonol . . . | C22H34O7 |
| tirofiban; Aggrastat; Tirofiban [BAN:INN] . . . | C22H36N2O5S |
| misoprostol; Cytotec; Arthrotec . . . | C22H38O5 |
| amikacin; Amicacin; Amikavet . . . | C22H43N5O13 |
| 1-DOCOSANOL; Behenyl alcohol; Docosanol . . . | C22H46O |
| Brequinar; 96187-53-0; brequinarum [Latin] . . . | C23H15F2NO2 |
| sulfinpyrazone; Sulphinpyrazone; Anturane . . . | C23H20N2O3S |
| chlorotrianisene; Chlortrianisestrol; Chlortrianizen . . . | C23H21ClO3 |
| TASOSARTAN; 145733-36-4; Verdia . . . | C23H21N7O |
| topotecan; Hycamtin; Hycamptamine . . . | C23H23N3O5 |
| doxazosin; Doxazosinum; Doxazosina . . . | C23H25N5O5 |
| Aripiprazole; Abilify; Abilitat . . . | C23H27Cl2N3O2 |
| risperidone; Risperdal; Risperidal . . . | C23H27FN4O2 |
| Paliperidone; Invega; 9-Hydroxyrisperidone . . . | C23H27FN4O3 |
| butenafine; Butenafina; Butenafinum . . . | C23H27N |
| piperacillin; Pipracil; Pipercillin . . . | C23H27N5O7S |
| glyburide; Glibenclamide; Glybenclamide . . . | C23H28ClN3O5S |
| Cinalukast; CHEBL126598; Cinalukast [USAN:INN] . . . | C23H28N2O3S |
| Forasartan; Forasartan (USAN/INN); UNII-065F7WPT0B . . . | C23H28N8 |
| cisapride; Propulsid; Acenalin . . . | C23H29ClFN3O4 |
| Tiotixene; Navane; cis-Thiothixene . . . | C23H29N3O2S2 |
| mepacrine; quinacrine; atabrine . . . | C23H30ClN3O |
| propantheline; Propanthelinium; Propanthelinum . . . | C23H30NO3+ |
| Methadyl acetate; Acetylmethadol; ALPHACETYLMETHADOL . . . | C23H31NO2 |
| Levacetylmethadol; Levomethadyl acetate; LAAM . . . | C23H31NO2 |
| NORGESTIMATE; Dexnorgestrel acetime; Ortho-Prefest . . . | C23H31NO3 |
| mycophenolate mofetil; CellCept; Munoloc . . . | C23H31NO7 |
| ramipril; Tritace; Altace . . . | C23H32N2O5 |
| Vardenafil; Levitra; 224785-90-4 . . . | C23H32N6O4S |
| finasteride; Proscar; Propecia . . . | C23H36N2O2 |
| pravastatin; Pravastatine [French]; Pravastatinum [Latin] . . . | C23H36O7 |
| paromomycin; Aminosidin; Paramomycin Sulfate . . . | C23H45N5O14 |
| neomycin; NEOMYCIN B; Framycetin . . . | C23H46N6O13 |
| candesartan; Biopress; 139481-59-7 . . . | C24H20N6O3 |
| Ezetimibe; Zetia; Ezetrol . . . | C24H21F2NO3 |
| eribaxaban; PD-0348292; 536748-46-6 . . . | C24H22ClFN4O4 |
| Aleglitazar; Aleglitazar (USAN); 475479-34-6 . . . | C24H23NO5S |
| flavoxate; Urispas; Flavoxato . . . | C24H25NO4 |
| nicergoline; Nicotergoline; Nimergoline . . . | C24H26BrN3O3 |
| SERTINDOLE; SerLect; Serdolect . . . | C24H26ClFN4O |
| carvedilol; Coreg; Carvedilolum . . . | C24H26N2O4 |
| troglitazone; Rezulin; Prelay . . . | C24H27NO5S |
| benazepril; Benazeprilum [Latin]; benazapril . . . | C24H28N2O5 |
| Bexarotene; Targretin; Targrexin . . . | C24H28O2 |
| valsartan; Diovan; 137862-53-4 . . . | C24H29N5O3 |
| donepezil; Eranz; 120014-06-4 . . . | C24H29NO3 |
| FLUOCINOLONE ACETONIDE; Synandone; Fluonid . . . | C24H30F2O6 |
| doxapram; Dopram; 309-29-5 . . . | C24H30N2O2 |
| Drospirenone; Dihydrospirorenone; Dehydrospirorenone . . . | C24H30O3 |
| Loteprednol etabonate; Lotemax; Alrex . . . | C24H31ClO7 |
| flunisolide; Nasalide; Aerobid . . . | C24H31FO6 |
| Carphenazine; Carfenazine; Carphenazin . . . | C24H31N3O2S |
| ETHYNODIOL DIACETATE; Continuin; Femulen . . . | C24H32O4 |
| spironolactone; Aldactone; Verospiron . . . | C24H32O4S |
| DESONIDE; DesOwen; Tridesilon . . . | C24H32O6 |
| RANOLAZINE; Ranexa; 95635-55-5 . . . | C24H33N3O4 |
| bepridil; Bepadin; Vascor . . . | C24H34N2O |
| trandolapril; Mavik; Gopten . . . | C24H34N2O5 |
| glimepiride; Amaryl; Amarel . . . | C24H34N4O5S |
| Org-6216; Vexol (TN); AC1NSKKX . . . | C24H34O3 |
| Nabilone; Cesamet; 51022-71-0 . . . | C24H36O3 |
| lovastatin; mevinolin; Mevacor . . . | C24H36O5 |
| dipyridamole; Persantin; Curantyl . . . | C24H40N8O4 |
| chenodeoxycholic acid; Chenodiol; Chenix . . . | C24H40O4 |
| ursodiol; URSODEOXYCHOLIC acid; Actigall . . . | C24H40O4 |
| Apixaban; 503612-47-3; Eliquis . . . | C25H25N5O4 |
| meclizine; Meclozine; Parachloramine . . . | C25H27ClN2 |
| irbesartan; Avapro; Aprovel . . . | C25H28N6O |
| amiodarone; Cordarone; Amiodarona . . . | C25H29I2NO3 |
| quinapril; Quinaprilum [Latin]; Quinaprilum . . . | C25H30N2O5 |
| FLUTICASONE PROPIONATE; Cutivate; Flonase . . . | C25H31F3O5S |
| CLOBETASOL PROPIONATE; Clobex; Embeline . . . | C25H32ClFO5 |
| nefazodone; Nefazodonum [Latin]; Nefazodona [Spanish] . . . | C25H32ClN5O2 |
| QUINESTROL; Estrovis; Estrovister . . . | C25H32O2 |
| amprenavir; Agenerase; Prozei . . . | C25H35N3O6S |
| Fosamprenavir; Telzir; Fosamprenavir [INN] . . . | C25H36N3O9PS |
| Udenafil; Zydena; DA-8159 . . . | C25H36N6O4S |
| Salmeterol; Astmerole; Serevent . . . | C25H37NO4 |
| Bimatoprost; Lumigan; Latisse . . . | C25H37NO4 |
| flunarizine; Sibelium; Flunarizinum [INN-Latin] . . . | C26H26F2N2 |
| IDARUBICIN; Idamycin; idarubicin hydrochloride . . . | C26H27NO9 |
| Ketoderm; Panfungol; Orifungal M . . . | C26H28Cl2N4O4 |
| Zuclomiphene; Cisclomiphene; Zuclomifene . . . | C26H28ClNO |
| toremifene; Acapodene; Farestone . . . | C26H28ClNO |
| cinnarizine; Stugeron; Dimitronal . . . | C26H28N2 |
| ALMITRINE; Almitrin; Vectarion . . . | C26H29F2N7 |
| levocabastine; Livostin; Levocabastinum [Latin] . . . | C26H29FN2O2 |
| nicardipine; Nicardipinum [INN-Latin]; Nicardipino [INN-Spanish] . . . | C26H29N3O6 |
| tamoxifen; Crisafeno; Diemon . . . | C26H29NO |
| Halofantrine; Halfan; Halofantrine [USAN] . . . | C26H30Cl2F3NO |
| terconazole; Triaconazole; Terazol . . . | C26H31Cl2N5O3 |
| Diflorasone diacetate; Florone; Psorcon . . . | C26H32F2O7 |
| fluocinonide; Lidex; Fluocinolide . . . | C26H32F2O7 |
| cerivastatin; Lipobay; cerivastatin acid . . . | C26H34FNO5 |
| Travoprost; Travatan; Travatan Z . . . | C26H35F3O6 |
| cabergoline; Dostinex; Cabaser . . . | C26H37N5O2 |
| Mupirocin; Pseudomonic acid; Bactroban . . . | C26H44O9 |
| clofazimine; Lamprene; Lampren . . . | C27H22Cl2N4 |
| Bosentan; Tracleer; 147536-97-8 . . . | C27H29N5O6S |
| daunorubicin; Daunomycin; Rubidomycin . . . | C27H29NO10 |
| doxorubicin; Adriamycin; Adriblastina . . . | C27H29NO11 |
| Gliquidone; Glurenorm; glikvidon . . . | C27H33N3O6S |
| MOEXIPRIL; Moexiprilum [INN-Latin]; 103775-10-6 . . . | C27H34N2O7 |
| Locorten; FLU METHASONE PIVALATE; Locacorten . . . | C27H36F2O6 |
| Repaglinide; Prandin; NovoNorm . . . | C27H36N2O4 |
| Darunavir; Prezista; TMC-114 . . . | C27H37N3O7S |
| VERAPAMIL; Iproveratril; Cordilox . . . | C27H38N2O4 |
| Paricalcitol; Zemplar; 19-Nor-1alpha,25-dihydroxyvitamin D2 . . . | C27H44O3 |
| raloxifene; Keoxifene; Raloxifenum [Latin] . . . | C28H27NO4S |
| ADAPALENE; Differin; Adaferin . . . | C28H28O3 |
| pimozide; Orap; Opiran . . . | C28H29F2N3O |
| Darifenacin; Emselex; CHEBL391960 . . . | C28H30N2O2 |
| astemizole; Hismanal; Alermizol . . . | C28H31FN4O |
| Buclizine; Vibazine; Histabuticine . . . | C28H33ClN2 |
| amcinonide; Cyclocort; amcinonida . . . | C28H35FO7 |
| Plerixafor; Mozobil; AMD3100 . . . | C28H54N8 |
| Lapatinib; Tykerb; 231277-92-2 . . . | C29H26ClFN4O4S |
| Olmesartan medoxomil; Benicar; 144689-63-4 . . . | C29H30N6O6 |
| Fluspirilene; Redeptin; Imap . . . | C29H31F2N3O |
| Imatinib; 152459-95-5; sti-571 . . . | C29H31N7O |
| etoposide; VePesid; Lastet . . . | C29H32O13 |
| Ioperamide; Ioperamide; Loperacap . . . | C29H33ClN2O2 |
| mifepristone; Mifeprex; Mifegyne . . . | C29H35NO2 |
| Mibefradil; Posicor; Mibefradil [INN:BAN] . . . | C29H38FN3O3 |
| Maraviroc; Celsentri; Selzentry . . . | C29H41F2N5O |
| Candoxatril; CHEBL3353; AC1NSFYL . . . | C29H41NO7 |
| orlistat; Xenical; Tetrahydrolipstatin . . . | C29H53NO5 |
| lividomycin A; Lividomycin; Quintomycin B . . . | C29H55N5O18 |
| DIPHENOXYLATE; Difenossilato [DCIT]; Difenoxilato . . . | C30H32N2O2 |
| Aliskiren; Rasilez; Tekturna . . . | C30H53N3O6 |
| Tipranavir; Aptivus; PNU-140690 . . . | C31H33F3N2O5S |
| zafirlukast; Accolate; 107753-78-6 . . . | C31H33N3O6S |
| Doramapimod; BIRB-796; BIRB 796 . . . | C31H37N5O3 |
| probucol; Lorelco; Biphenabid . . . | C31H48O2S2 |
| fusidic acid; Fusidine; Ramycin . . . | C31H48O6 |
| Conivaptan; YM 087; YM087 . . . | C32H26N4O2 |
| DESERPIDINE; Harmonyl; Raunormine . . . | C32H38N2O8 |
| fexofenadine; Carboxyterfenadine; Terfenadine-COOH . . . | C32H39NO4 |
| bromocriptine; Bromocriptin; Bromocryptine . . . | C32H40BrN5O5 |
| terfenadine; Seldane; Triludan . . . | C32H41NO2 |
| nelfinavir; 159989-64-7; AG1343 . . . | C32H45N3O4S |
| Fulvestrant; Faslodex; ICI 182,780 . . . | C32H47F5O3S |
| Telmisartan; Micardis; Pritor . . . | C33H30N4O2 |
| atorvastatin; Cardyl; Sotis . . . | C33H35FN2O5 |
| Ergotamin; Ergostat; Gynergen . . . | C33H35N5O5 |
| dihydroergotamine; Ergomimet; Orstanorm . . . | C33H37N5O5 |
| irinotecan; Camptosar; (+)-Irinotecan . . . | C33H38N4O6 |
| reserpine; Serpasil; Serpalan . . . | C33H40N2O9 |
| montelukast; Montair; Singular . . . | C35H36ClNO3S |
| itraconazole; Sporanox; Oriconazole . . . | C35H38Cl2N8O4 |
| rescinnamine; Rescinnamine; Moderil . . . | C35H42N2O9 |
| ENALKIREN; Enalquireno; Abbott-64662 . . . | C35H56N6O6 |
| Lercanidipine; Lercanidipine [INN]; masnidipine . . . | C36H41N3O6 |
| indinavir; Compound J; Crixivan . . . | C36H47N5O4 |
| tubocurarine; d-Tubocurarine; Tubocurarin . . . | C37H41N2O6+ |
| Lopinavir; Aluviran; ABT-378 . . . | C37H48N4O5 |
| ritonavir; Norvir; Norvir Sec . . . | C37H48N6O5S2 |
| erythromycin; erythromycin A; Erymax . . . | C37H67NO13 |
| Atazanavir; Latazanavir; Zrivada . . . | C38H52N6O7 |
| digitoxin; Digitoxoside; Crystodigin (TN) . . . | C41H64O13 |
| TROLEANDOMYCIN; Triacetyloleandomycin; Matromicina . . . | C41H67NO15 |
| Leucomycin A3; Turimycin A5; josamycin . . . | C42H69NO15 |
| Rifaximin; Rifaxidin; Xifaxan . . . | C43H51N3O11 |
| docetaxel; Taxotere; Docetaxel anhydrous . . . | C43H53NO14 |
| vindesine; Desacetylvinblastine amide; Vindesin . . . | C43H55N5O7 |
| vinorelbine; Navelbine base; Navelbine . . . | C45H54N4O8 |
| vincristine; Leurocristine; 22-Oxovincaleukoblastine . . . | C46H56N4O10 |
| RIFABUTIN; Ansamycin; Mycobutin . . . | C46H62N4O11 |
| TAXOL; paclitaxel; Abraxane . . . | C47H51NO14 |
| Priftin; RIFAPENTINE; Cyclopentyl rifampin . . . | C47H64N4O12 |
| amphotericin b; Amphotericin; Ambisome . . . | C47H73NO17 |
| Terlipressin; glypressin; Lucassin . . . | C52H74N16O15S2 |
| Anidulafungin; Eraxis; Ecalta . . . | C58H73N7O17 |
| Mivacurium; MIVACURIUM CHLORIDE; AC1NQXXZ . . . | C58H80N2O14 + 2 |

1. A method of treating a disease state, comprising administering to patient in need thereof, a pharmaceutically effective amount of a compound that is derived from plant materials and comprises at least 50 mass % bio-based carbons; and wherein the compound is Cetirizine, Chlorhexidine (chlorhexamed forte), Ambroxol (mucosolvan), Bisacodyl (Ducolax), Xylomethazoline (Olynth), Diclofenac (forte voltaren), Clotrimazole (canesten), Omeprazole (omep Hexal), Flurbiprofen (Dobendan), Naproxen (Dolormin), Doxilamine (Hoggar), Ioperamide, Ibuprofen, Fexofenadine, or Paracetamol.

2. The method of claim 1 wherein 12 or 13 of the 20 carbon atoms in cetirizine are bio-based.

3. The method of claim 1 wherein the cetirizine is administered in multiple doses over at least 5 days in a dosage regimen, and wherein patient compliance is increased by at least 20%.

4-5. (canceled)

6. A pharmaceutically active compound that is at least partially derived from biomass.

7. The pharmaceutically active compound of claim 6, comprising at least one aromatic group and wherein the at least one aromatic group of the compound is derived from biomass.

8. The pharmaceutically active compound of claim 7, wherein the entire compound is derived from biomass.

9. The pharmaceutically active compound of claim 6 having a 14C:12C isotopic ratio that is similar to the 14C:12C isotopic ratio of a living organism.

10. The pharmaceutically active compound of claim 6 selected from the list of Table 1 having a 14C content of approximately 1 part per trillion.

11. The compound of claim 6, wherein the compound is lansoprazole or mephentermine.

12. (canceled)

13. A pharmaceutical composition comprising at least one compound of claim 6.

14. The pharmaceutical composition of claim 13, further comprising at least one pharmaceutically acceptable excipient.

15. A pharmaceutical composition of claim 13, comprising at least two pharmaceutically active compounds.

16. A method of treating a disease state comprising administering to patient in need thereof, a composition of claim 13.

17-18. (canceled)

19. The method of n claim 16, wherein the patient knows that the at least one pharmaceutically active compound is at least partially derived from biomass.

20. The method of claim 19, wherein patient compliance is improved.

21. A method of improving patient compliance with a pharmaceutical dosing regime comprising including administering a pharmaceutically active compound that is at least partially derived from biomass in the dosing regime.

22. The method of claim 21 wherein the dosing regime comprises multiple doses over at least 3 days.

23. (canceled)

24. The pharmaceutically active compound of claim 6 in which between 10 and 90 mass % of the carbon atoms are bio-based.

25-27. (canceled)

28. The pharmaceutically active compound of claim 6 wherein the compound is Cetirizine, Chlorhexidine (chlorhexamed forte), Ambroxol (mucosolvan), Bisacodyl (Ducolax), Xylomethazoline (Olynth), Diclofenac (forte voltaren), Clotrimazole (canesten), Omeprazole (omep Hexal), Flurbiprofen (Dobendan), Naproxen (Dolormin), Doxilamine (Hoggar), Ioperamide, Ibuprofen, Fexofenadine, or Paracetamol.

29. (canceled)

30. The compound of claim 6 wherein the compound is: Chlorhexidine in which 12 of the 22 carbons are bio-based, Ambroxol in which 7 of the 13 carbons are bio-based, Bisacodyl in which 12 or 16 of the 22 carbons are bio-based, Xylomethazoline in which 8 or 10 of the 14 carbons are bio-based, Diclofenac in which 13 of the 14 carbons are bio-based, Clotrimazole in which 17 of the 20 carbons are bio-based, Omeprazole in which 6 of the 17 carbons are bio-based, Flurbiprofen in which 6 or 12 of the 15 carbons are bio-based, Naproxen in which 10 or 11 of the 14 carbons are bio-based, Doxilamine in which 6 or 10 of the 17 carbons are bio-based, or Ioperamide in which 16, 18 or 22 of the 29 carbons are bio-based.

31. (canceled)
